(19) United States
(12) Patent Application Publication
(10) Pub. No.: US 2011/0144576 A1
(43) Pub. Date: Jun. 16, 2011

(54) CATHETER ORIENTATION CONTROL SYSTEM MECHANISMS

(75) Inventors: Chris A. ROTHE, San Mateo, CA (US); Vahid SAADAT, Atherton, CA (US)

(73) Assignee: Voyage Medical, Inc., Redwood City, CA (US)

(21) Appl. No.: 12/967,288

(22) Filed: Dec. 14, 2010

Related U.S. Application Data
(60) Provisional application No. 61/286,283, filed on Dec. 14, 2009.
(60) Provisional application No. 61/297,462, filed on Jan. 22, 2010.

(51) Int. Cl. A61M 25/092

(52) U.S. Cl. 604/95.04; 604/95.01

(57) ABSTRACT

Catheter orientation control system mechanisms which facilitate the operation of multiple degrees of freedom of a steerable catheter system can be used for any procedure where catheter orientation relative to the body is desirable. Such systems may comprise a handle having a distal portion which is rotatable independently of the remainder of the handle to enable the handle to maintain a stationary configuration relative to the operator while the catheter distal end is torqued in any configuration by utilizing a single hand and/or single finger to effect complex configurations. Orientation indicators may be incorporated to track the deflectable distal end by visualizing the indicators through an imaging hood on the distal end corresponding to orientation markers on the control handle. Articulation of a steering mechanism in a direction relative to the orientation markers deflects the catheter distal end in a corresponding direction relative to the visualized orientation markers.

### CROSS-REFERENCE TO RELATED APPLICATIONS

This application claims the benefit of priority to U.S. Provisional Application 61/286,283 filed Dec. 14, 2009 and 61/297,462 filed Jan. 22, 2010, each of which is incorporated herein by reference in its entirety.

### FIELD OF THE INVENTION

The present invention relates generally to catheter control systems for controlling the articulation of visualization and treatment apparatus having imaging and manipulation features for intravascularly accessing regions of the body.

### BACKGROUND OF THE INVENTION

Conventional devices for accessing and visualizing interior regions of a body lumen are known. For example, various catheter devices are typically advanced within a patient's body, e.g., intravascularly, and advanced into a desirable position within the body. Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, many of the conventional catheter imaging systems lack the capability to provide therapeutic treatments or are difficult to manipulate in providing effective therapies. For instance, the treatment in a patient's heart for atrial fibrillation is generally made difficult by a number of factors, such as visualization of the target tissue, access to the target tissue, and instrument articulation and management, amongst others.

Conventional catheter techniques and devices, for example such as those described in U.S. Pat. Nos. 5,895,417; 5,941,845; and 6,129,724, used on the epicardial surface of the heart may be difficult in assuring a transmural lesion or complete blockage of electrical signals. In addition, current devices may have difficulty dealing with varying thickness of tissue through which a transmural lesion is desired.

Conventional accompanying imaging devices, such as fluoroscopy, are unable to detect perpendicular electrode orientation, catheter movement during the cardiac cycle, and image catheter position throughout lesion formation. The absence of real-time visualization also poses the risk of incorrect placement and ablation of structures such as sinus node tissue which can lead to fatal consequences.

Moreover, because of the tortuous nature of intravascular access, devices or mechanisms at the distal end of a catheter positioned within the patient's body, e.g., within a chamber of the heart, are typically no longer aligned with the handle. Steering or manipulation of the distal end of the catheter via control or articulation mechanisms on the handle is easily disorienting to the user as manipulation of a control on the handle in a first direction may articulate the catheter distal end in an unexpected direction depending upon the resulting catheter configuration leaving the user to adjust accordingly. However, this results in reduced efficiency and longer procedure times as well as increased risks to the patient. Accordingly, there is a need for improved catheter control systems which facilitate the manipulation and articulation of a catheter.

### BRIEF SUMMARY OF THE INVENTION

A tissue imaging and manipulation apparatus that may be utilized for procedures within a body lumen, such as the heart, in which visualization of the surrounding tissue is made difficult, if not impossible, by medium contained within the lumen such as blood, is described below. Generally, such a tissue imaging and manipulation apparatus comprises an optional delivery catheter or sheath through which a deployment catheter and imaging hood may be advanced for placement against or adjacent to the tissue to be imaged.

The deployment catheter may define a fluid delivery lumen therethrough as well as an imaging lumen within which an optical imaging fiber or assembly may be disposed for imaging tissue. When deployed, the imaging hood may be expanded into any number of shapes, e.g., cylindrical, conical as shown, semi-spherical, etc., provided that an open area or field is defined by the imaging hood. The open area is the area within which the tissue region of interest may be imaged. The imaging hood may also define an atraumatic contact lip or edge for placement or abutment against the tissue region of interest. Moreover, the distal end of the deployment catheter or separate manipulatable catheters may be articulated through various controlling mechanisms such as push-pull wires manually or via computer control

The deployment catheter may also be stabilized relative to the tissue surface through various methods. For instance, inflatable stabilizing balloons positioned along a length of the catheter may be utilized, or tissue engagement anchors may be passed through or along the deployment catheter for temporary engagement of the underlying tissue.

In operation, after the imaging hood has been deployed, fluid may be pumped at a positive pressure through the fluid delivery lumen until the fluid fills the open area completely and displaces any blood from within the open area. The fluid may comprise any biocompatible fluid, e.g., saline, water, plasma, Fluorinert™, etc., which is sufficiently transparent to allow for relatively undistorted visualization through the fluid. The fluid may be pumped continuously or intermittently to allow for image capture by an optional processor which may be in communication with the assembly.

In an exemplary variation for imaging tissue surfaces within a heart chamber containing blood, the tissue imaging and treatment system may generally comprise a catheter body having a lumen defined therethrough, a visualization element disposed adjacent the catheter body, the visualization element having a field of view, a transparent fluid source in fluid communication with the lumen, and a barrier or membrane extendable from the catheter body to localize, between the visualization element and the field of view, displacement of blood by transparent fluid that flows from the lumen, and an instrument translatable through the displaced blood for performing any number of treatments upon the tissue surface within the field of view. The imaging hood may be formed into any number of configurations and the imaging assembly may also be utilized with any number of therapeutic tools which may be deployed through the deployment catheter.

More particularly in certain variations, the tissue visualization system may comprise components including the imaging hood, where the hood may further include a membrane having a main aperture and additional optional openings disposed over the distal end of the hood. An introducer sheath or the deployment catheter upon which the imaging hood is disposed may further comprise a steerable segment made of multiple adjacent links which are pivotably connected to one another and which may be articulated within a single plane or multiple planes. The deployment catheter itself may be comprised of a multiple lumen extrusion, such as a four-lumen catheter extrusion, which is reinforced with braided stainless steel fibers to provide structural support. The proximal end of the catheter may be coupled to a handle for manipulation and articulation of the system.

To provide visualization, an imaging element such as a fiberscope or electronic imager such as a solid state camera, e.g., CCD or CMOS, may be mounted, e.g., on a shape memory wire, and positioned within or along the hood interior. A fluid reservoir and/or pump (e.g., syringe, pressurized intravenous bag, etc.) may be fluidly coupled to the proximal end of the catheter to hold the translucent fluid such as saline or contrast medium as well as for providing the pressure to inject the fluid into the imaging hood.

One example of a system configured to enable direct visualization of tissue underlying the hood and optionally treat tissue, e.g., ablation, may include an ablation assembly, hood, and deployment catheter coupled to a handle having a catheter steering assembly integrated along the handle. The steering handle assembly may enable a user to steer the visualization hood along at least two or more planes in multiple degrees of freedom relative to a longitudinal axis of the catheter. The handle assembly may include a handle portion and articulation housing which may extend at an angle proximally relative to the handle portion to position a distal steering control, e.g., having an articulation control member extending from the control, readily within the reach of the operator's thumb when his/her hand is gripped about the handle portion. The articulation control may be configured as a projection (such as a joystick) extending from distal steering control for facilitating manipulation by the operator; however, the articulation control may be configured in any number of shapes in alternative configurations to facilitate the control of the distal steering control by the operator's finger or fingers. By manipulating the control, e.g., with a single finger such as the operator's thumb, the distal steerable section may be articulated in any number directions, e.g., at least two or more different planes, relative to the catheter to control the articulation of the hood.

The handle may also incorporate a proximal steering control which may be rotated about the handle portion to actuate a proximal steering portion, e.g., located proximal to the distal steering portion, to articulate the proximal steering portion in at least one plane in either direction by rotating the control in either direction correspondingly. Although described as a rotatable control member, the proximal steering control may be alternatively actuated through any number of different mechanisms, e.g., levers, triggers, etc. Manipulating or pulling along a portion of the distal steering control causes the steerable portion and the hood to move along a corresponding direction of articulation. Moreover, because of the manner in which the articulation housing is positioned to extend along the angled housing from the handle portion, the operator may grip the handle and operate the handle assembly with a single hand.

Additionally, a distal handle portion may extend from the articulation housing for attachment to the catheter. The distal handle portion may be shaped in the configuration shown as a tapered nosecone tapering distally towards the catheter attachment, however, the distal handle portion may be shaped in any number of other configurations. Moreover, the distal handle portion may be attached to the articulation housing via a rotatable coupling which may allow for the handle portion to rotate about its longitudinal axis relative to the remainder of the handle to allow for the catheter and hood to be rotated during advancement and positioning within the patient body while allowing for the articulation housing to remain in a stationary position relative to the operator.

The proximal steering control may be actuated, e.g., by rotating the control in a first direction, to articulate the proximal steerable section within a first plane, e.g., to retroflex the hood and the distal steerable section in a corresponding direction of articulation. The hood may be further articulated by manipulating the articulation control of the distal steering control, e.g., in a direction of actuation, such that distal steerable section moves in a corresponding direction of articulation. The steering control may be further actuated in another direction of articulation to move the distal steerable section and hood in a corresponding direction of articulation while maintaining the proximal steerable section in its configuration. In one variation, the proximal steerable section may be configured to articulate via the proximal steering control within a single plane while distal steerable section may be configured to articulate in at least four directions. However, both the proximal steering control and distal steering control can be manipulated in varying degrees to steer the respective steerable sections to varying curvatures as desired by the operator.

As previously mentioned, the design of the catheter handle assembly allows the operator to easily grasp the assembly with a single hand, left or right hand, and articulate either or both the proximal steering control and/or the distal steering control with a single finger, e.g., the operator's thumb (although any of the operator's fingers may be utilized as desirable). This enables a single operator to effectively control full articulation of the catheter and hood (or any other distal end effector) through multiple degrees-of-freedom within a patient body with a single hand and/or a single finger.

Although multiple pullwires may be utilized in the control handle depending upon the number of directions for articulation, four pullwires may be typically utilized. Each of the four pullwires may be terminated symmetrically around a circumference of the steering control such that a balanced four-way steering of the distal portion may be accomplished, although manipulating the steering control along various portions of its circumference may yield combinational articulation between the pullwires to result in numerous catheter configurations. Additionally, the handle assembly may further incorporate a spring mechanism as an overdrive prevention mechanism. The spring mechanism may be positioned between the transition manifold and steering control in order to prevent over-tensioning or breaking of the pullwires if the steering control is over-deflected in a direction.

In utilizing the multi-articulation steering with the proximal steering control and/or distal steering control, a distal handle portion may be attached to the articulation housing via a rotatable coupling which may allow for the handle portion to rotate about its longitudinal axis relative to the remainder of the handle. The one or more pullwires coupled to the steering control may pass through the angled housing and into a torquing section defined within and/or between the articulation housing and distal handle portion. The distal handle portion may be rotated in one or both directions about a longitudinal axis of the handle assembly. With the catheter attached securely to the distal handle portion, as the hood and catheter is advanced through the patient's body intravascularly and, e.g., into the chambers of the heart, the distal end of the catheter having the hood may be articulated into a tortuous configuration.

The operator imaging the tissue regions through the hood may become disoriented when steering the catheter in a particular desired direction. This could result in reorienting the handle assembly in a configuration, e.g., upside down relative to the operator's position, making steering and articulation of the catheter awkward given the positioning of the controls along the handle. Thus, the distal handle portion may be rotated about the coupling to accommodate any rotation and orientation of the catheter while enabling the remainder of the handle portion and articulation housing to remain in a constant configuration relative to the operator. Moreover, the one or more pullwires may become twisted over one another within the torquing section. Because the wires may be encased in respective isolating structures or isolation coils, e.g., compression coils, they may twist upon one another while still remaining free to translate through the coils to effectively transmit the appropriate tension to articulate the distal steerable section. The distal handle portion may be rotated relative to the articulation housing by up to 720 degrees or more while still allowing for the one or more pullwires to sufficiently transmit the tension for articulation. Alternatively, a stop may be incorporated between the distal handle portion to limit its rotation relative to the articulation housing to prevent over-torquing of the pullwires, e.g., limiting rotation up to 270 degrees in one or both rotational directions.

With the distal handle portion and catheter being rotatable relative to the remainder of the handle assembly, the catheter and hood can be consistently deflected in the same direction by which the steering controls are being deflected regardless of the orientation of the handle assembly. For example, the handle assembly may be deflected in a first direction of actuation such that the hood is deflected in a corresponding first direction of articulation. The distal handle portion, catheter, and hood are then rotated along an arbitrary direction of rotation about the longitudinal axis of the handle assembly while maintaining a constant position of the handle assembly relative to the operator. Even with the distal handle portion rotated, e.g., 180°, actuating the steering controls along a direction of actuation still results in a corresponding direction of articulation of the hood which matches the first direction of articulation despite the rotated assembly. Regardless of the angle by which the operator subsequently rotates the catheter about the longitudinal axis, the operator can still be certain that deflecting the steering controls in a particular direction will steer the distal end of the catheter in the same direction. This removes the need for the operator to memorize the original position of the catheter or how much the catheter has been torqued in order to gauge the orientation of the deflected end when the catheter is inserted into the patient and further prevents the handle assembly from becoming oriented in an awkward position relative to the operator.

Once the distal handle portion has been rotated to re-orient a configuration of the hood within the patient's body, various mechanisms may be utilized for locking and maintaining a position of the rotated handle portion relative to the handle housing. The ability to lock and unlock a position of the distal handle portion relative to the housing may allow for the operator to ensure that the re-oriented hood and catheter will maintain its configuration within the patient's body without fear of releasing or becoming displaced inadvertently. Moreover, the various controls on the handle assembly, such as the articulation control, one-way steering controls, distal handle portion, etc., may be selectively locked and/or unlocked via various mechanisms.

Additionally and/or alternatively, visual indicators positioned directly upon the hood may also be utilized in coordination with corresponding visual indicators positioned upon the distal steering control. The hood may have one or more visual indicators marked upon the distal portion of the hood such that the visual image on the monitor as captured through the hood may show at least a first directional indicator along a first portion of the hood. In this example, a second directional indicator and yet a third corresponding third indicator and fourth directional indicator may be positioned about a circumference of the hood or hood membrane to represent any number of directions. The handle assembly may thus have one or more directional indicators located directly upon, e.g., the distal steering control, which is shown in this variation as a circular configuration and which corresponds spatially with the indicators positioned upon the hood or hood membrane. For instance, the first directional indicator on the hood may correspond spatially with the first directional indicator on the distal steering control, second directional indicator on the hood may correspond spatially with the second directional indicator on the distal steering control, third directional indicator on the hood may correspond with the third directional indicator on the distal steering control, and the fourth directional indicator on the hood may correspond with the fourth directional indicator on the distal steering control, and so on. Although four directional indicators are shown in this example, fewer than four or more than four may be utilized. Moreover, the location and positioning of the indicators may also be varied, as desired. Additionally, each of the directional indicators may be color-coded by different colors and/or shapes or symbols to distinguish between the different directions.

In use, the directional indicators as viewed through the hood correspond to the direction the hood may move when the distal steering control is deflected along the position where the corresponding indicator is located. Thus, deflecting the distal steering control in a direction of actuation, e.g., along any one or combination of the directional indicators, may articulate the distal steerable section and hood in a corresponding direction of articulation along the directional indicator shown on the hood or hood membrane. This removes complexity in steering the hood, e.g., when the hood is in a retroflexed position, where directions are reversed with respect to the operator.

Yet another variation of the catheter handle may include a handle assembly comprising a support housing which is removably engagable to a receiving handle. Having the support housing removable from the receiving handle may allow for the operator to initially insert and advance the catheter and its distal end effector, such as the hood, with the handle assembly as a complete assembly. If desired, the operator may then disengage the support housing from the receiving handle to provide unconstrained freedom in articulating the steerable section via the distal controller while allowing for the catheter to remain stationary relative to the patient's body.

Additionally and/or alternatively, the catheter handle assembly may be removably attached to a platform secured to a bed, railing or directly to the patient's own body, for securing a position of the catheter and handle relative to the entry point into the patient's body. This platform may have one or more adjustable features to accommodate the handle assembly as well as adjusting the platform position relative to the patient's body.

The catheter control systems described herein may additionally integrate any number of features and controls for facilitate procedures. These features and controls may be integrated into any of the variations described herein. One example may include features such as flow rate control, air bubble detection, ablation activation switches, built-in image sensors, etc., may be incorporated into the handle assembly.

### BRIEF DESCRIPTION OF THE DRAWINGS

FIG. 1A shows a side view of one variation of a tissue imaging apparatus during deployment from a sheath or delivery catheter.

FIG. 1B shows the deployed tissue imaging apparatus of FIG. 1A having an optionally expandable hood or sheath attached to an imaging and/or diagnostic catheter.

FIG. 1C shows an end view of a deployed imaging apparatus.

FIGS. 2A and 2B show one example of a deployed tissue imager positioned against or adjacent to the tissue to be imaged and a flow of fluid, such as saline, displacing blood from within the expandable hood.

FIGS. 3A and 3B show examples of various visualization imagers which may be utilized within or along the imaging hood.

FIGS. 4A and 4B show perspective and end views, respectively, of an imaging hood having at least one layer of a transparent elastomeric membrane over the distal opening of the hood.

FIGS. 5A and 5B show perspective and end views, respectively, of an imaging hood which includes a membrane with an aperture defined therethrough and a plurality of additional openings defined over the membrane surrounding the aperture.

FIGS. 6A to 6C show perspective assembly views of one variation of a steering handle assembly which is configure to enable a user to steer a visualization hood along at least four or more degrees of freedom relative to a longitudinal axis of the catheter.

FIGS. 7A to 7D show perspective views of a catheter control handle configured to articulate a proximal and distal steerable section.

FIG. 7E shows a perspective view of a distal portion of the catheter control handle rotated relative to a proximal portion of the catheter control handle to re-orient the hood and steerable sections.

FIGS. 8A to 8D show perspective views of the catheter control handle of FIGS. 7A to 7D manipulated via a single hand and/or single finger of the operator.

FIGS. 9A and 9B show perspective views of the distal steering assembly normally enclosed within the handle assembly.

FIGS. 10A and 10B show perspective exploded and assembly views of the distal steering control.

FIGS. 11A to 11D show cross-sectional side views of the catheter control handle illustrating the rotatable coupling and torquing section for the one or more pullwires.

FIGS. 12A and 12B show perspective views of a catheter control handle illustrating the transition manifold for the one or more pullwires.

FIGS. 13A to 13C show perspective and detail perspective exploded assembly views of the handle assembly.

FIGS. 14A and 14B show another variation of the catheter control handle and a corresponding view through the imaging hood illustrating the one or more directional indicators.

FIGS. 14C and 14E show perspective views of the catheter control handle attached to the deployment catheter with hood and steerable sections.

FIGS. 15A to 15C show side and cross-sectional side views of a variation of the distal catheter handle incorporating a locking feature.

FIGS. 16A to 16C show perspective and cross-sectional views of another variation of the distal catheter handle incorporating a dis-engagable locking feature.

FIGS. 17A and 17B show perspective views of the articulation control which incorporates a locking feature to maintain a catheter configuration.

FIGS. 18A to 18C show perspective views of another variation of an articulation control which incorporates a locking feature.

FIGS. 19A and 19B show perspective views of yet another variation of an articulation control which incorporates a locking feature.

FIGS. 20A and 20B show perspective views of yet another variation of an articulation control which incorporates a locking feature.

FIGS. 21A to 21C show another variation of the catheter handle having a support housing which is removably engagable to a receiving handle.

FIGS. 22A to 22C show side and cross-sectional side views of the catheter handle and a detachable support housing variation incorporating imaging and steering controls.

FIGS. 23A and 23B show perspective views of a detachable catheter handle variation which may be supported upon a railing or table platform.

FIGS. 24A and 24B show perspective and top views of a platform which may be used to temporarily secure a catheter control handle relative to a patient's body during a procedure.

FIG. 24C shows a top view of another variation of a platform which may be translated and/or rotated relative to the patient's body in a secure manner.

FIGS. 25A to 25C show perspective views of platform variations which may be secured directly to a patient, to a railing or table platform, or simply laid atop a patient during use.

FIG. 26 shows a perspective view of a variation where a catheter control handle may be attached to the user.

### DETAILED DESCRIPTION OF THE INVENTION

A tissue-imaging and manipulation apparatus described herein is able to provide real-time images in vivo of tissue regions within a body lumen such as a heart, which is filled with blood flowing dynamically therethrough and is also able to provide intravascular tools and instruments for performing various procedures upon the imaged tissue regions. Such an apparatus may be utilized for many procedures, e.g., facilitating transseptal access to the left atrium, cannulating the coronary sinus, diagnosis of valve regurgitation/stenosis, valvuloplasty, atrial appendage closure, arrhythmogenic focus ablation, among other procedures.

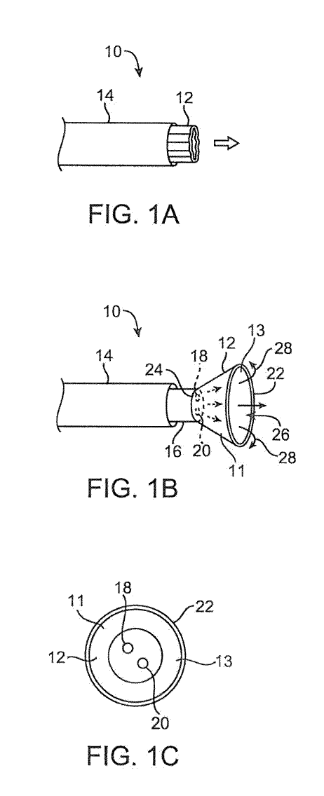

One variation of a tissue access and imaging apparatus is shown in the detail perspective views of FIGS. 1A to 1C. As shown in FIG. 1A, tissue imaging and manipulation assembly 10 may be delivered intravascularly through the patient's body in a low-profile configuration via a delivery catheter or sheath 14. In the case of treating tissue, it is generally desirable to enter or access the left atrium while minimizing trauma to the patient. To non-operatively effect such access, one conventional approach involves puncturing the intra-atrial septum from the right atrial chamber to the left atrial chamber in a procedure commonly called a transseptal procedure or septostomy. For procedures such as percutaneous valve repair and replacement, transseptal access to the left atrial chamber of the heart may allow for larger devices to be introduced into the venous system than can generally be introduced percutaneously into the arterial system.

When the imaging and manipulation assembly 10 is ready to be utilized for imaging tissue, imaging hood 12 may be advanced relative to catheter 14 and deployed from a distal opening of catheter 14, as shown by the arrow. Upon deployment, imaging hood 12 may be unconstrained to expand or open into a deployed imaging configuration, as shown in FIG. 1B. Imaging hood 12 may be fabricated from a variety of pliable or conformable biocompatible material including but not limited to, e.g., polymeric, plastic, or woven materials. One example of a woven material is Kevlar® (E. I. du Pont de Nemours, Wilmington, Del.), which is an aramid and which can be made into thin, e.g., less than 0.001 in., materials which maintain enough integrity for such applications described herein. Moreover, the imaging hood 12 may be fabricated from a translucent or opaque material and in a variety of different colors to optimize or attenuate any reflected lighting from surrounding fluids or structures, i.e., anatomical or mechanical structures or instruments. In either case, imaging hood 12 may be fabricated into a uniform structure or a scaffold-supported structure, in which case a scaffold made of a shape memory alloy, such as Nitinol, or a spring steel, or plastic, etc., may be fabricated and covered with the polymeric, plastic, or woven material. Hence, imaging hood 12 may comprise any of a wide variety of barriers or membrane structures, as may generally be used to localize displacement of blood or the like from a selected volume of a body lumen or heart chamber. In exemplary embodiments, a volume within an inner surface 13 of imaging hood 12 will be significantly less than a volume of the hood 12 between inner surface 13 and outer surface 11.

Imaging hood 12 may be attached at interface 24 to a deployment catheter 16 which may be translated independently of deployment catheter or sheath 14. Attachment of interface 24 may be accomplished through any number of conventional methods. Deployment catheter 16 may define a fluid delivery lumen 18 as well as an imaging lumen 20 within which an optical imaging fiber or assembly may be disposed for imaging tissue. When deployed, imaging hood 12 may expand into any number of shapes, e.g., cylindrical, conical as shown, semi-spherical, etc., provided that an open area or field 26 is defined by imaging hood 12. The open area 26 is the area within which the tissue region of interest may be imaged. Imaging hood 12 may also define an atraumatic contact lip or edge 22 for placement or abutment against the tissue region of interest. Moreover, the diameter of imaging hood 12 at its maximum fully deployed diameter, e.g., at contact lip or edge 22, is typically greater relative to a diameter of the deployment catheter 16 (although a diameter of contact lip or edge 22 may be made to have a smaller or equal diameter of deployment catheter 16). For instance, the contact edge diameter may range anywhere from 1 to 5 times (or even greater, as practicable) a diameter of deployment catheter 16. FIG. 1C shows an end view of the imaging hood 12 in its deployed configuration. Also shown are the contact lip or edge 22 and fluid delivery lumen 18 and imaging lumen 20.

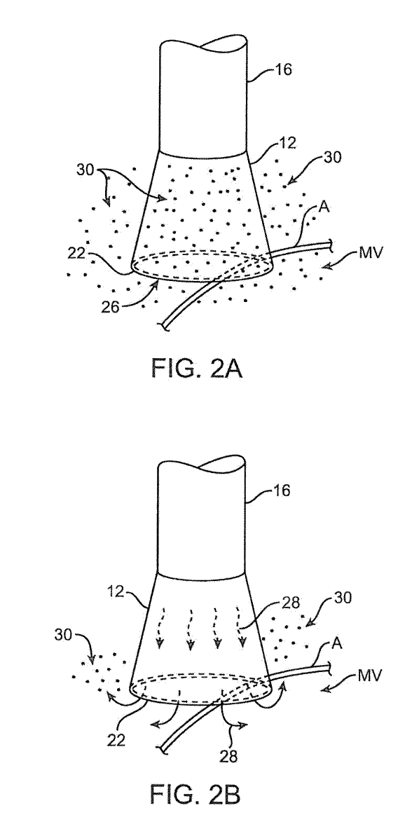

As seen in the example of FIGS. 2A and 2B, deployment catheter 16 may be manipulated to position deployed imaging hood 12 against or near the underlying tissue region of interest to be imaged, in this example a portion of annulus A of mitral valve MV within the left atrial chamber. As the surrounding blood 30 flows around imaging hood 12 and within open area 26 defined within imaging hood 12, as seen in FIG. 2A, the underlying annulus A is obstructed by the opaque blood 30 and is difficult to view through the imaging lumen 20. The translucent fluid 28, such as saline, may then be pumped through fluid delivery lumen 18, intermittently or continuously, until the blood 30 is at least partially, and preferably completely, displaced from within open area 26 by fluid 28, as shown in FIG. 2B.

Although contact edge 22 need not directly contact the underlying tissue, it is at least preferably brought into close proximity to the tissue such that the flow of clear fluid 28 from open area 26 may be maintained to inhibit significant backflow of blood 30 back into open area 26. Contact edge 22 may also be made of a soft elastomeric material such as certain soft grades of silicone or polyurethane, as typically known, to help contact edge 22 conform to an uneven or rough underlying anatomical tissue surface. Once the blood 30 has been displaced from imaging hood 12, an image may then be viewed of the underlying tissue through the clear fluid 30. This image may then be recorded or available for real-time viewing for performing a therapeutic procedure. The positive flow of fluid 28 may be maintained continuously to provide for clear viewing of the underlying tissue. Alternatively, the fluid 28 may be pumped temporarily or sporadically only until a clear view of the tissue is available to be imaged and recorded, at which point the fluid flow 28 may cease and blood 30 may be allowed to seep or flow back into imaging hood 12. This process may be repeated a number of times at the same tissue region or at multiple tissue regions.

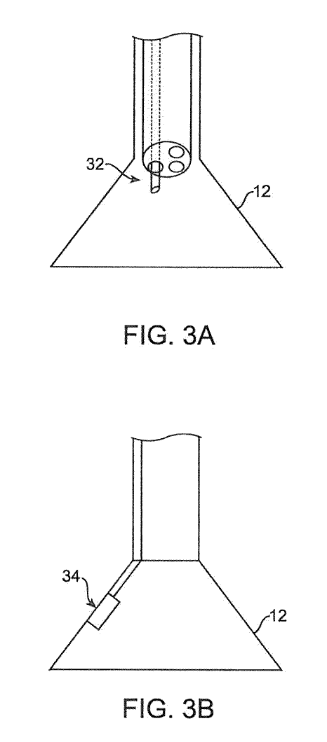

FIG. 3A shows a partial cross-sectional view of an example where one or more optical fiber bundles 32 may be positioned within the catheter and within imaging hood 12 to provide direct in-line imaging of the open area within hood 12. FIG. 3B shows another example where an imaging element 34 (e.g., CCD or CMOS electronic imager) may be placed along an interior surface of imaging hood 12 to provide imaging of the open area such that the imaging element 34 is off-axis relative to a longitudinal axis of the hood 12, as described in further detail below. The off-axis position of element 34 may provide for direct visualization and uninhibited access by instruments from the catheter to the underlying tissue during treatment.

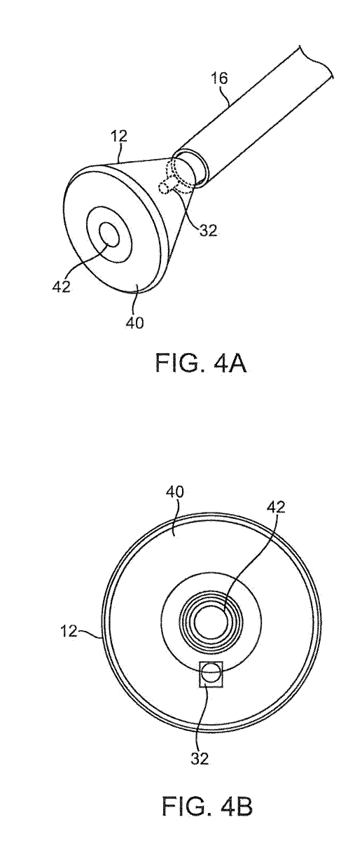

In utilizing the imaging hood 12 in any one of the procedures described herein, the hood 12 may have an open field which is uncovered and clear to provide direct tissue contact between the hood interior and the underlying tissue to effect any number of treatments upon the tissue, as described above. Yet in additional variations, imaging hood 12 may utilize other configurations. An additional variation of the imaging hood 12 is shown in the perspective and end views, respectively, of FIGS. 4A and 4B, where imaging hood 12 includes at least one layer of a transparent elastomeric membrane 40 over the distal opening of hood 12. An aperture 42 having a diameter which is less than a diameter of the outer lip of imaging hood 12 may be defined over the center of membrane 40 where a longitudinal axis of the hood intersects the membrane such that the interior of hood 12 remains open and in fluid communication with the environment external to hood 12. Furthermore, aperture 42 may be sized, e.g., between 1 to 2 mm or more in diameter and membrane 40 can be made from any number of transparent elastomers such as silicone, polyurethane, latex, etc. such that contacted tissue may also be visualized through membrane 40 as well as through aperture 42.

Aperture 42 may function generally as a restricting passageway to reduce the rate of fluid out-flow from the hood 12 when the interior of the hood 12 is infused with the clear fluid through which underlying tissue regions may be visualized. Aside from restricting out-flow of clear fluid from within hood 12, aperture 42 may also restrict external surrounding fluids from entering hood 12 too rapidly. The reduction in the rate of fluid out-flow from the hood and blood in-flow into the hood may improve visualization conditions as hood 12 may be more readily filled with transparent fluid rather than being filled by opaque blood which may obstruct direct visualization by the visualization instruments.

Moreover, aperture 42 may be aligned with catheter 16 such that any instruments (e.g., piercing instruments, guidewires, tissue engagers, etc.) that are advanced into the hood interior may directly access the underlying tissue uninhibited or unrestricted for treatment through aperture 42. In other variations wherein aperture 42 may not be aligned with catheter 16, instruments passed through catheter 16 may still access the underlying tissue by simply piercing through membrane 40.

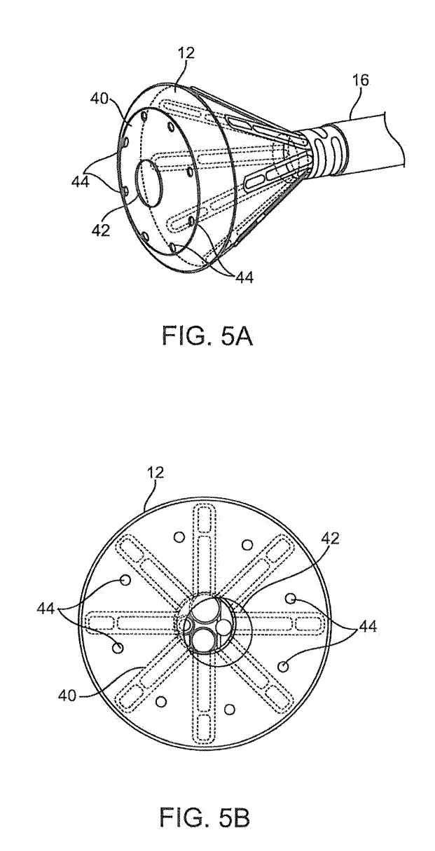

In an additional variation, FIGS. 5A and 5B show perspective and end views, respectively, of imaging hood 12 which includes membrane 40 with aperture 42 defined therethrough, as described above. This variation includes a plurality of additional openings 44 defined over membrane 40 surrounding aperture 42. Additional openings 44 may be uniformly sized, e.g., each less than 1 mm in diameter, to allow for the out-flow of the translucent fluid therethrough when in contact against the tissue surface. Moreover, although openings 44 are illustrated as uniform in size, the openings may be varied in size and their placement may also be non-uniform or random over membrane 40 rather than uniformly positioned about aperture 42 in FIG. 5B. Furthermore, there are eight openings 44 shown in the figures although fewer than eight or more than eight openings 44 may also be utilized over membrane 40.

Additional details of tissue imaging and manipulation systems and methods which may be utilized with apparatus and methods described herein are further described, for example, in U.S. patent application Ser. No. 11/259,498 filed Oct. 25, 2005 (U.S. Pat. Pub. 2006/0184048 A1), which is incorporated herein by reference in its entirety.

In utilizing the devices and methods above, various procedures may be accomplished. One example of such a procedure is crossing a tissue region such as in a transseptal procedure where a septal wall is pierced and traversed, e.g., crossing from a right atrial chamber to a left atrial chamber in a heart of a subject. Generally, in piercing and traversing a septal wall, the visualization and treatment devices described herein may be utilized for visualizing the tissue region to be pierced as well as monitoring the piercing and access through the tissue. Details of transseptal visualization catheters and methods for transseptal access which may be utilized with the apparatus and methods described herein are described in U.S. patent application Ser. No. 11/763,399 filed Jun. 14, 2007 (U.S. Pat. Pub. 2007/0293724 A1), which is incorporated herein by reference in its entirety. Additionally, details of tissue visualization and manipulation catheter which may be utilized with apparatus and methods described herein are described in U.S. patent application Ser. No. 11/259,498 filed Oct. 25, 2005 (U.S. Pat. Pub. 2006/0184048 A1), which is incorporated herein by reference in its entirety.

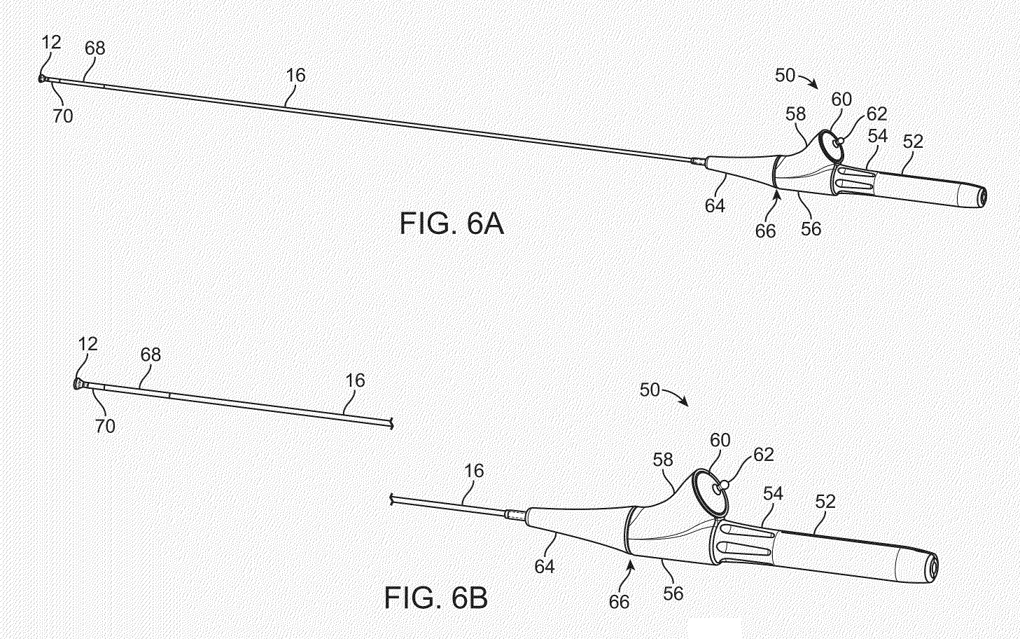

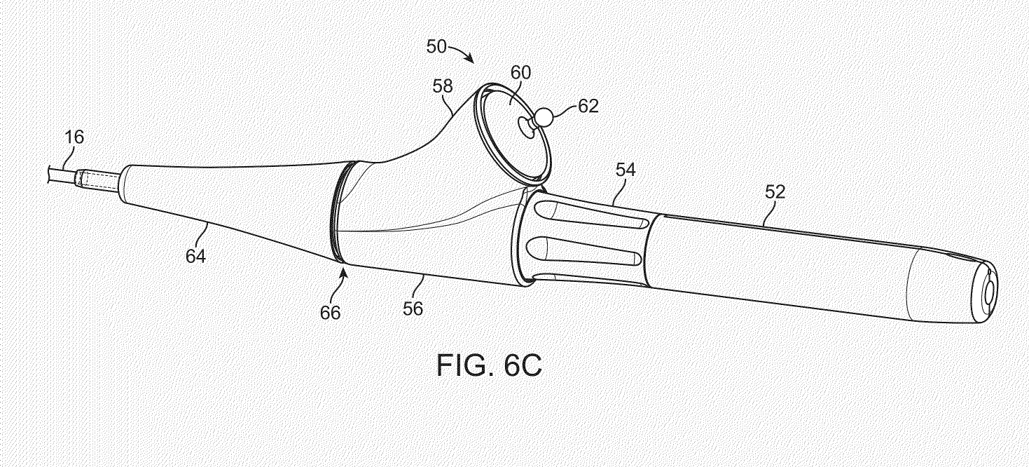

FIGS. 6A to 6C show assembly views of one variation of a steering handle assembly 50 which enables a user to steer the visualization hood 12 along at least four or more degrees of freedom relative to a longitudinal axis of the catheter 16. The handle assembly 50 is illustrated with handle portion 52 and articulation housing 56 which may extend at an angle proximally relative to handle portion 52 to position a distal steering control 60, e.g., having an articulation control 62 extending from control 60, readily within the reach of the operator's thumb when his/her hand is gripped about handle portion 52. Thus, the distal steering control 60 may be angled to be off-set relative to the proximal steering control 54 such that the distal control 60 is adjacent to the proximal control 54. Articulation control 62 is shown in this example as a projection (such as a joystick) extending from distal steering control 60 for facilitating manipulation by the operator; however, articulation control 62 may be configured in any number of shapes in alternative configurations to facilitate the control of distal steering control 60 by the operator's finger or fingers. By manipulating control 60, e.g., with a single finger such as the operator's thumb, distal steerable section 70 may be articulated in any number directions, e.g., at least two or more different planes, relative to catheter 16 to control the articulation of the hood 12. Handle 50 may also incorporate a proximal steering control 54 which may be rotated about handle portion 52 to actuate a proximal steering portion 68, e.g., located proximal to the distal steering portion 70, to articulate the proximal steering portion 68 in at least one plane in either direction by rotating the control 54 in either direction correspondingly. Although shown and described as a rotatable control member, the proximal steering control may be alternatively actuated through any number of different mechanisms, e.g., levers, triggers, etc. Manipulating or pulling along a portion of distal steering control 60 causes steerable portion 70 and hood 12 to move along a corresponding direction of articulation, as described in further detail below. Moreover, because of the manner in which articulation housing 56 is positioned to extend along angled housing 58 from the handle portion 52, the operator may grip the handle 50 and operate the handle assembly 50 with a single hand. Moreover, although the controls may be optimized for manipulation and articulation via a single finger, multiple fingers may be utilized in various combinations if so desired. However, with the design optimized, a single finger may be used to manipulate the device.

Additionally, a distal handle portion 64 may extend from articulation housing 56 for attachment to catheter 16. Distal handle portion 64 is shaped in the configuration shown as a tapered nosecone tapering distally towards the catheter attachment, however, distal handle portion 64 may be shaped in any number of other configuration. Moreover, distal handle portion 64 may be attached to articulation housing 56 via a rotatable coupling 66 which may allow for handle portion 64 to rotate about its longitudinal axis relative to the remainder of the handle, as described in further detail below, to allow for catheter 16 and hood 12 to be rotated during advancement and positioning within the patient body while allowing for the articulation housing 56 to remain in a stationary position relative to the operator.

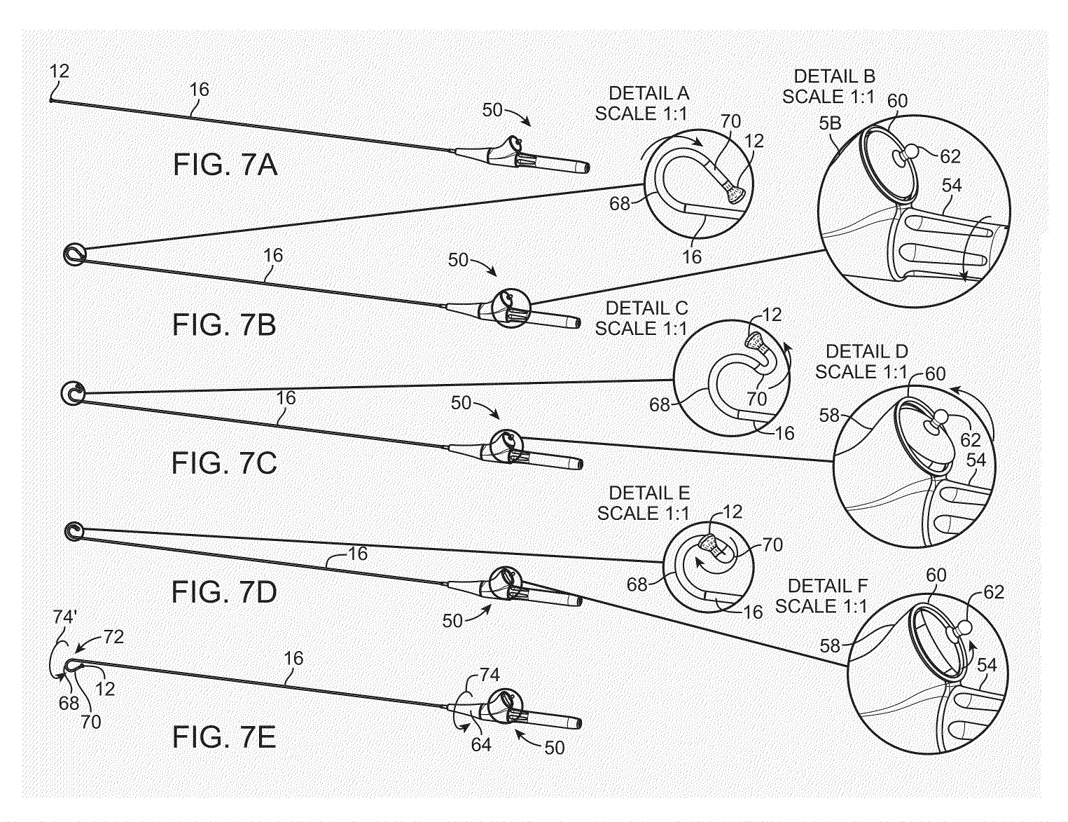

This particular handle assembly 50 may be used to control articulation of the hood 12 and the distal steerable section 70 as well as used to further control articulation of the proximal steerable section 68. As shown in the perspective views of FIGS. 7A to 7D, proximal steering control 54 may be actuated, e.g., by rotating the control 54 in a first direction, to articulate the proximal steerable section 68 within a first plane, e.g., to retroflex hood 12 and distal steerable section 70 in a corresponding direction of articulation, as shown in FIG. 7B. Hood 12 may be further articulated by manipulating articulation control 62 of distal steering control 60, e.g., in a direction of actuation, such that distal steerable section 70 moves in a corresponding direction of articulation, as shown in FIG. 7C. FIG. 7D illustrates how distal steering control 60 may be further actuated in another direction of articulation to move distal steerable section 70 and hood 12 in a corresponding direction of articulation while maintaining the proximal steerable section 68 in its configuration. In one variation, proximal steerable section 68 may be configured to articulate via proximal steering control 54 within a single plane while distal steerable section 70 may be configured to articulate in at least four directions, as described above. However, both the proximal steering control 54 and distal steering control 60 can be manipulated in varying degrees to steer the respective steerable sections to varying curvatures as desired by the operator.

As illustrated in FIG. 7E, prior to, during, or after hood 12 has been articulated to direct it to a particular tissue region within the patient's body, the distal handle portion 64 may be rotated relative to the remainder of the handle, as illustrated by the direction of rotation 74 of distal handle portion 64. Such rotation of portion 64 may correspondingly rotate catheter 16 about its own longitudinal axis even when curved to accordingly rotate the distal assembly 72, including hood 12 and steerable sections 68, 70, as indicated by the corresponding direction of rotation 74′, to redirect hood 12. Because distal handle portion 64 is coupled to the handle via a rotatable coupling 66, the distal handle portion 64 may be rotated while allowing for the catheter handle to remain in a stationary position relative to the operator thus enabling the operator to maintain an orientation of the handle as well as to maintain an orientation of the direction by which hood 12 may be articulated relative to the controls on the handle when viewed upon a monitor through the images captured through hood 12, as described in further detail below.

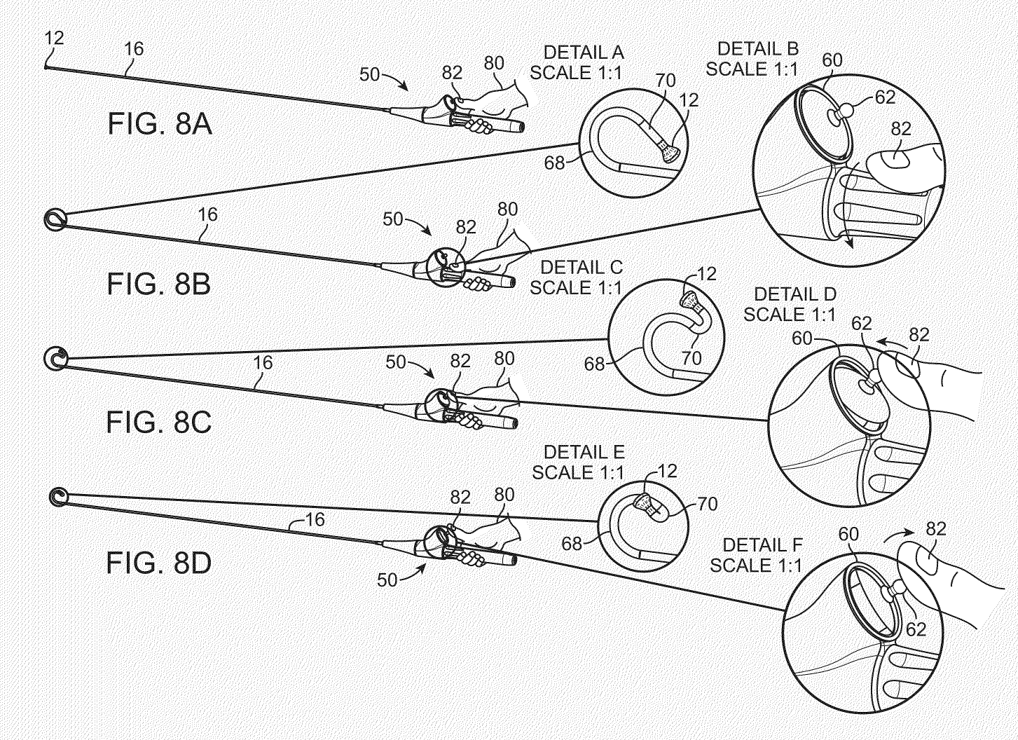

As previously mentioned, the design of the catheter handle assembly 50 allows the operator to easily grasp the assembly 50 with a single hand 80, left or right hand, and articulate either or both the proximal steering control 54 and/or the distal steering control 60 with a single finger, e.g., the operator's thumb 82 (although any of the operator's fingers may be utilized alone or in combination as desirable). This enables a single operator to effectively control full articulation of the catheter 16 and hood 12 (or any other distal end effector) through multiple degrees-of-freedom within a patient body with a single hand and/or a single finger. As illustrated in the perspective view of FIG. 8A, a single hand 80 of the operator (such as the right hand) may be seen grasping the handle assembly 50 for advancing catheter 16 and hood 12 within the patient's body.

As shown in FIG. 8B, at least one finger 82 (e.g., the operator's thumb) may be utilized to articulate proximal steering control 54 to actuate proximal steerable section 68. With the proximal steerable section 68 articulated, the single finger 82 of the operator may be used to articulate distal steering control 60 to then actuate distal steerable section 70 and hood 12, as shown in FIGS. 8C and 8D, in any number of configurations.

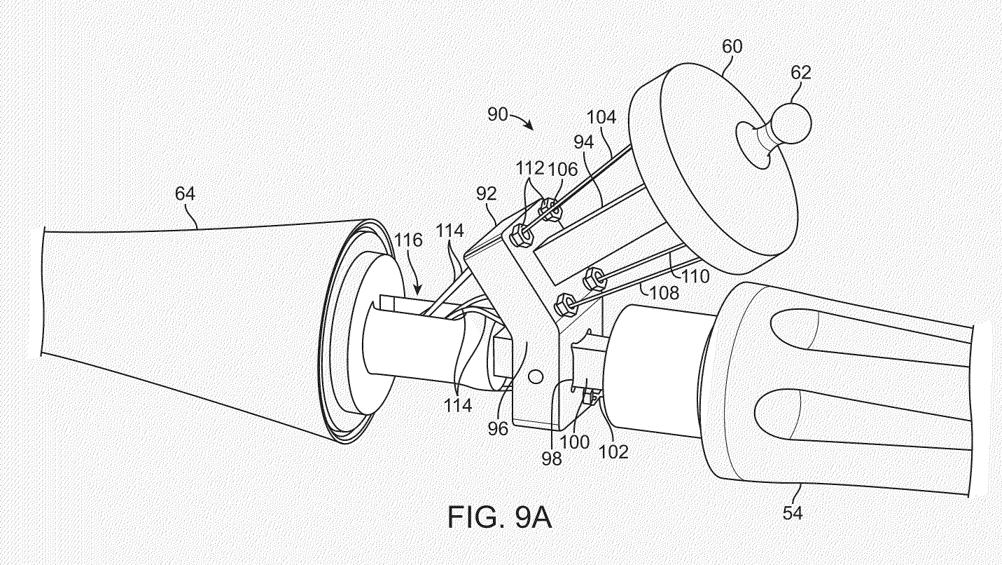

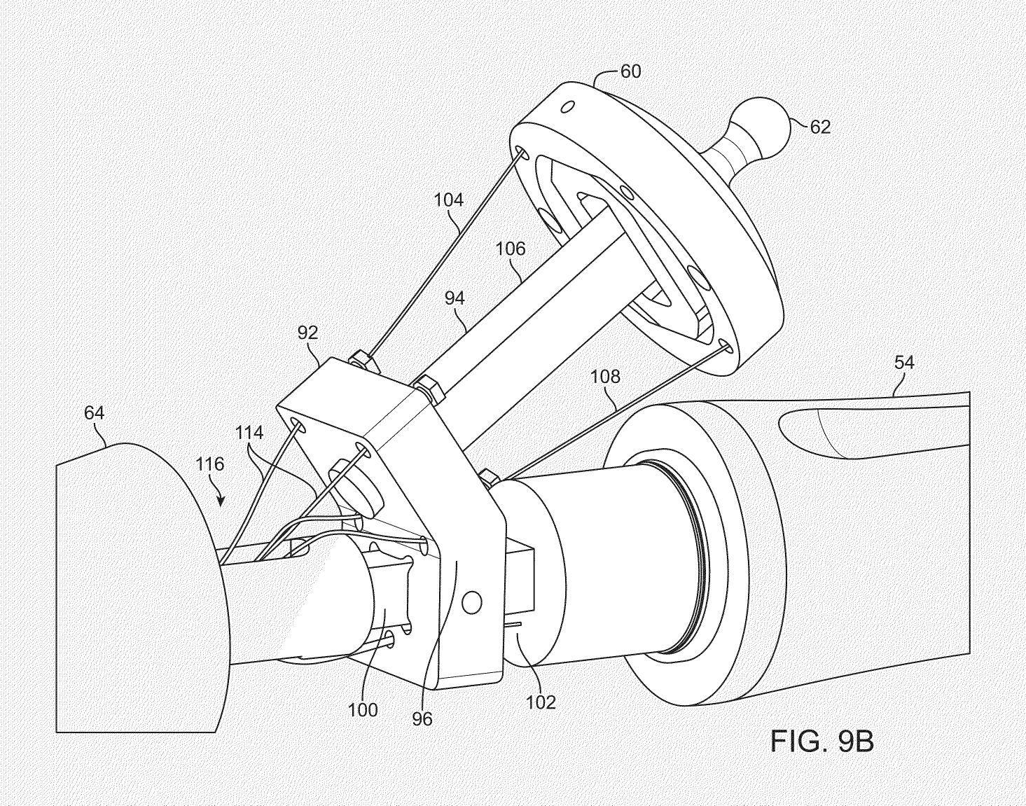

As shown in the perspective views of FIGS. 9A and 9B, handle assembly 50 may generally comprise a distal steering assembly 90 enclosed within the housing. Distal steering control 60 may be seen extending from platform support member 92 via a control support member 94 which provides a structural support element for holding steering control 60. Platform support member 92 may comprise an angled section 96 for holding control support member 94 and steering control 60 at an angle relative to the remainder of the handle, e.g., angled section 96 may have an angle of 30° relative to the platform support member 92. This angle may be varied depending upon the desired angle at which steering control 60 is to be positioned relative to the handle. Because platform support member 92 provides structural support to steering control 60, it may be secured to the handle assembly in part by a longitudinal support member 100, described in further detail below, passing through opening 98 defined through support member 92. Platform support member 98 may also define one or more channels, e.g., to accommodate the proximal steering control wire 102 coupled to proximal steering control 54.

Steering control 60 may be moved about control support member 94 in any number of directions to tension one or more pullwires 104, 106, 108, 110 for correspondingly controlling the distal steerable section 70. The terminal ends of the one or more pullwires 104, 106, 108, 110 may be coupled at circumferential locations uniformly about steering control 60 via corresponding fasteners, e.g., set screws, securing each of the pullwire termination crimps. These pullwires 104, 106, 108, 110 may extend through corresponding receiving channels 112, defined through platform support member 92, and through pullwire transition manifold 116 and into a proximal end of a multi-lumen shaft, such as catheter 16. The pullwires may continue distally through catheter 16 where they may be coupled to the distal steerable section 70. Each of the pullwires may be optionally encased in corresponding isolating structures or isolation coils, e.g., compression coils 114, through the transition manifold 116 between platform support member 92 and catheter 16.

Although multiple pullwires may be utilized depending upon the number of directions for articulation, four pullwires may be typically utilized. Each of the four pullwires may be terminated symmetrically around a circumference of steering control 60 such that a balanced four-way steering of the distal portion may be accomplished, although manipulating the steering control 60 along various portions of its circumference may yield combinational articulation between the pullwires to result in numerous catheter configurations. Additionally, the handle assembly 50 may further incorporate a spring mechanism as an overdrive prevention mechanism. The spring mechanism may be positioned between the transition manifold 116 and steering control 60 in order to prevent over-tensioning or breaking of the pullwires if the steering control 60 is over-deflected in a direction.

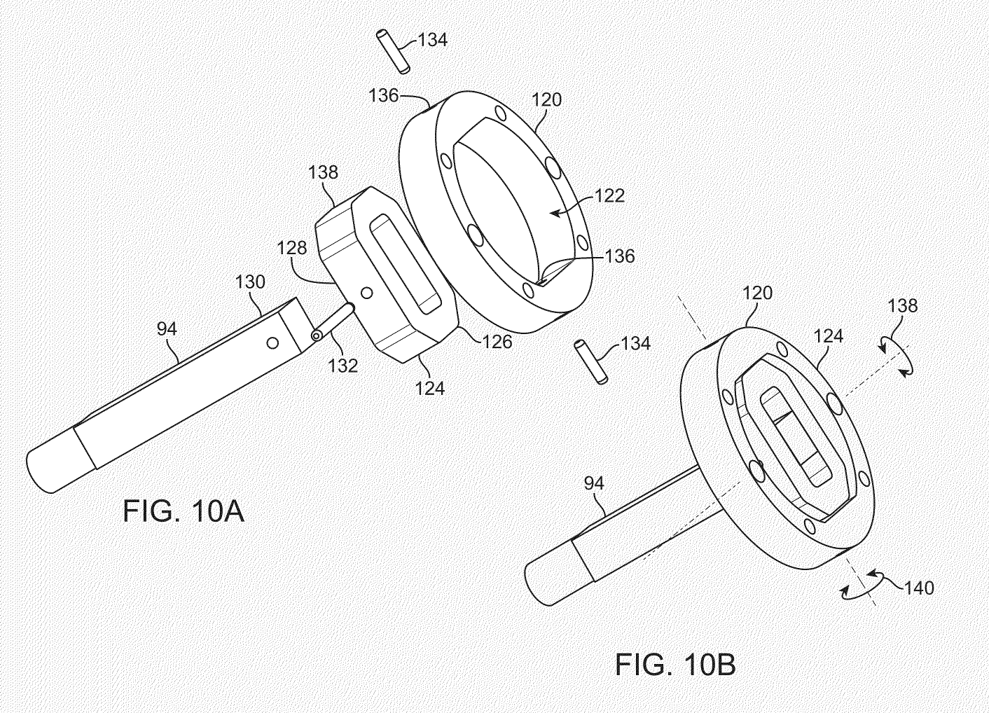

To enable the multiple directions of articulation with steering control 60, an example of the pivoting support is illustrated in the exploded and assembly perspective views of FIGS. 10A and 10B, respectively. In this example, a first support member 120 (e.g., configured into a circular shape) may define an opening 122 within which a second support member 124 may be positioned. Second support member 124 may be supported via one or more pins 124 on opposed ends passing through openings 136 on first support member 120 and openings 138 on second support member 122 such that when second support member 124 is positioned within opening 122 of first support member 120, second support member 124 may freely pivot about the pins 134 about a first axis. Second support member 124 may likewise be pivotably coupled to control support member 94 via one or more pins 132 passing through openings 128 on second support member 124 and openings 130 on control support member 94. The openings between second support member 124 and control support member 124 may be located transversely relative to the openings coupling first support member 120 to second support member 124. The assembly results in a control mechanism which allows for steering control 60, coupled to first support member 120, to be articulated about either a first rotational axis 138 and/or a second rotational axis 140 transverse to the first rotational axis 138, or a combination of both rotational axes 138, 140 to provide articulation of the distal steerable section 70 along multiple planes, as shown in FIG. 10B.

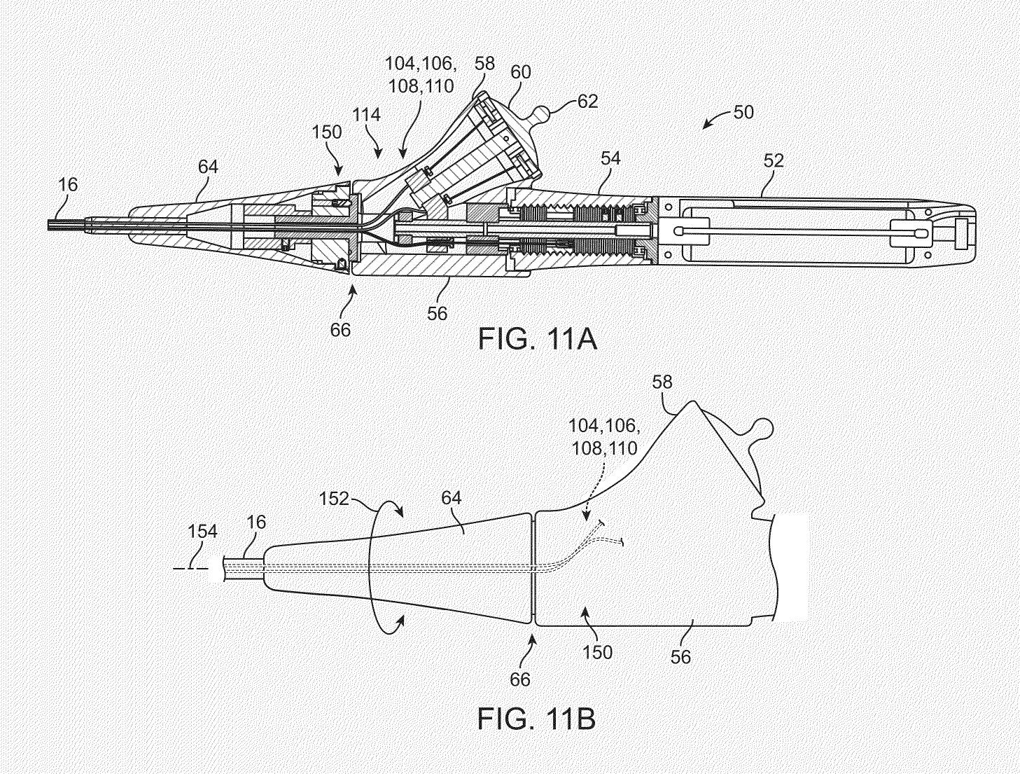

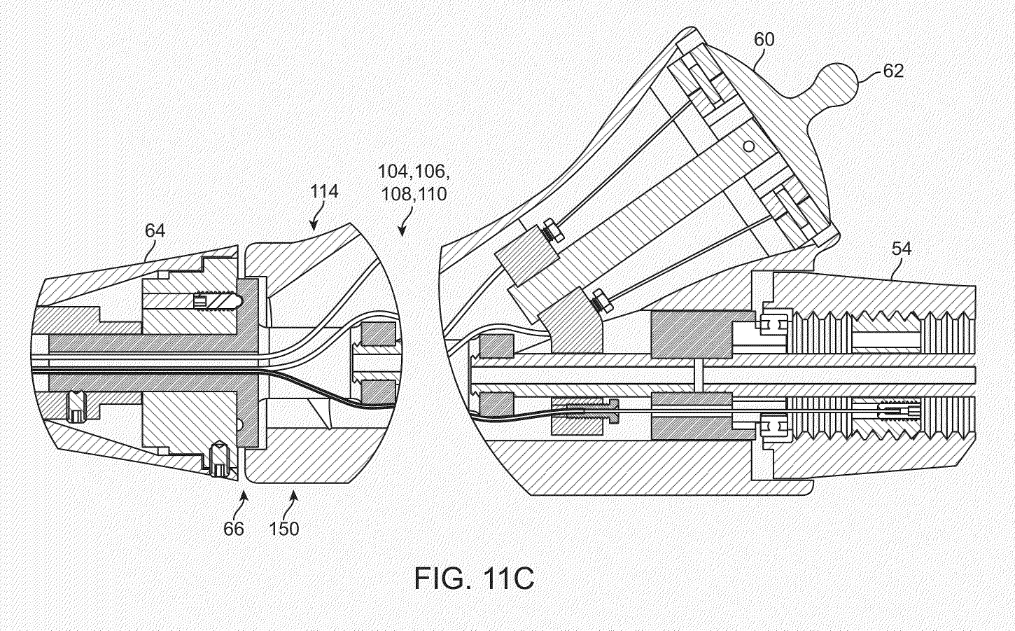

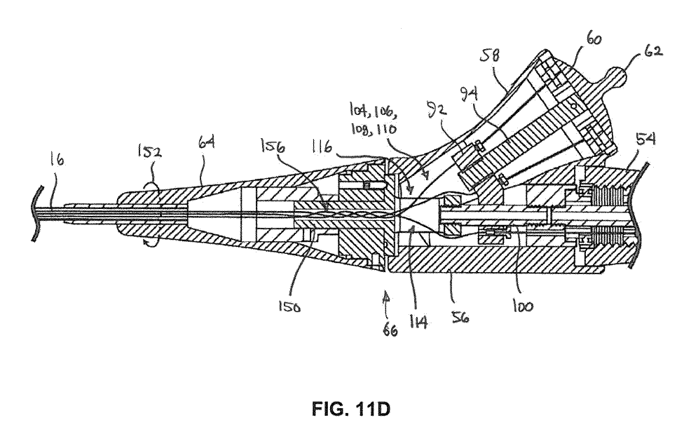

In utilizing the multi-articulation steering with the proximal steering control 54 and/or distal steering control 60, a distal handle portion 64 may be attached to articulation housing 56 via a rotatable coupling 66 which may allow for handle portion 64 to rotate about its longitudinal axis relative to the remainder of the handle. As shown in the cross-sectional side views of FIGS. 11A to 11C, the one or more pullwires 104, 106, 108, 110 coupled to steering control 60 may pass through angled housing 58 and into a torquing section 150 within a distal portion of the pullwire manifold 116 defined within and/or between articulation housing 56 and distal handle portion 64. As previously described, distal handle portion 64 may be rotated in one or both directions indicated by arrow 152 about a longitudinal axis 154 of handle assembly 50, as shown in FIG. 11B. With catheter 16 attached securely to distal handle portion 64, as hood 12 and catheter 16 is advanced through the patient's body intravascularly and, e.g., into the chambers of the heart, the distal end of the catheter 16 having hood 12 may be articulated into a tortuous configuration.

The operator imaging the tissue regions through hood 12 may become disoriented when steering the catheter in a particular desired direction. This could result in reorienting the handle assembly 50 in a configuration, e.g., upside down relative to the operator's position, making steering and articulation of the catheter awkward given the positioning of the controls along the handle 50. Thus, distal handle portion 64 may be rotated about coupling 66 to accommodate any rotation and orientation of catheter 16, as shown by arrow 152, while enabling the remainder of handle portion 52 and articulation housing 56 to remain in a constant configuration relative to the operator. Moreover, the one or more pullwires 104, 106, 108, 110 may become twisted over one another, as shown, within torquing section 150 of a pullwire transition manifold 116, as described in further detail below. Because the wires may be encased in respective isolation coils 114, they may twist upon one another while still remaining free to translate through the coils 114 to effectively transmit the appropriate tension to articulate the distal steerable section 70. The distal handle portion 64 may be rotated relative to articulation housing 56 by up to 720 degrees or more while still allowing for the one or more pullwires 104, 106, 108, 110 to sufficiently transmit the tension for articulation. Alternatively, a stop may be incorporated between distal handle portion 64 to limit its rotation relative to articulation housing 56 to prevent over-torquing of the pullwires, e.g., limiting rotation up to 270 degrees in one or both rotational directions.

FIG. 11D shows another cross-sectional side view illustrating the twisted isolation coils 156 twisted upon one another within the torquing section 150 of transition manifold 116 as the distal handle portion 64 is rotated relative to the handle in the direction 152. Because the twisted wires (and each respective isolation coil) are localized within or in proximity to the transition manifold 116, the lengths of the pullwires through the remainder of the handle assembly and through deployment catheter 16 may remain unperturbed by the twisting of distal handle portion 64 when re-orienting the hood and distal steerable section.

With distal handle portion 64 and catheter 16 being rotatable relative to the remainder of the handle assembly 50, catheter 16 and hood 12 can be consistently deflected in the same direction by which the steering controls 54, 60 are being deflected regardless of the orientation of the handle assembly 50. For example, handle assembly 50 may be deflected in a first direction of actuation such that hood 12 is deflected in a corresponding first direction of articulation. The distal handle portion 64, catheter 16, and hood 12 are then rotated along an arbitrary direction of rotation about longitudinal axis 154 of the handle assembly 50 while maintaining a constant position of handle assembly 50 relative to the operator. Even with the distal handle portion 64 rotated, e.g., 180°, actuating the steering controls 54, 60 along a direction of actuation still results in a corresponding direction of articulation of hood 12 which matches the first direction of articulation despite the rotated assembly. Regardless of the angle by which the operator subsequently rotates the catheter 16 about the longitudinal axis 154, the operator can still be certain that deflecting the steering controls 54, 60 in a particular direction will steer the distal end of the catheter in the same direction. This removes the need for the operator to memorize the original position of the catheter or how much the catheter has been torqued in order to gauge the orientation of the deflected end when the catheter is inserted into the patient and further prevents the handle assembly 50 from becoming oriented in an awkward position relative to the operator.

Utilizing such catheter steering may be particularly advantageous for tissue treatment, e.g., ablation, in the left atrium of the heart as such adaptability in steering may impart additional accuracy and efficiency to steer the imaging and ablation hood 12 around complex anatomical structures, such as the pulmonary vein ostium. Examples of such steerable catheters having any number of features which may be utilized with features described herein are shown and described in further detail in U.S. patent application Ser. No. 12/108,812 filed Apr. 24, 2008 (U.S. Pat. Pub. 2008/0275300 A1) and Ser. No. 12/117,655 filed May 8, 2008 (U.S. Pat. Pub. 2008/0281293 A1) and Ser. No. 12/499,011 filed Jul. 7, 2009, each of which is incorporated herein by reference in its entirety.

Moreover, these handle variations as well as any of the other handle variations herein may incorporate any of the features described in each of the variations, as practicable. For instance, this particular variation may also utilize the optical adjustment assembly, locking mechanisms, etc. in combination if so desired.

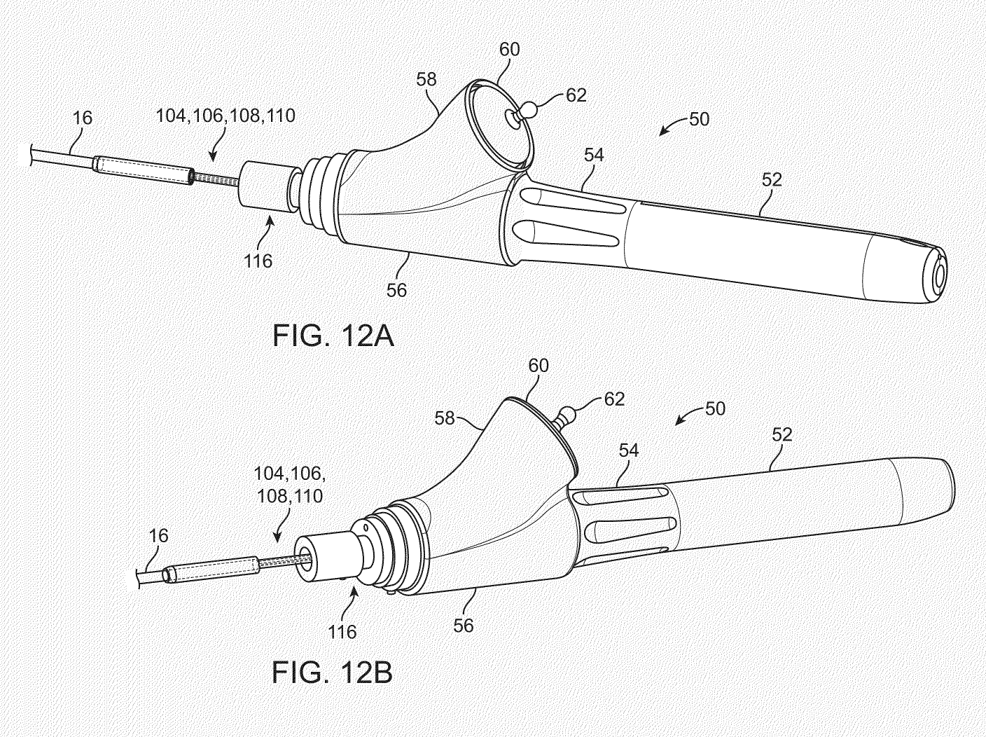

FIGS. 12A and 12B show perspective views of handle assembly 50 having distal handle portion 64 removed to illustrate the positioning between the transition manifold 116 and one or more pullwires 104, 106, 108, 110 relative to catheter 16. As shown, manifold 116 may extend from articulation housing 56 and at least partially into distal handle portion 64 such that the one or more pullwires 104, 106, 108, 110 may be twisted upon or over one another therewithin (within torquing section 150) when distal handle portion 64 is rotated relative to articulation housing 56 during use.

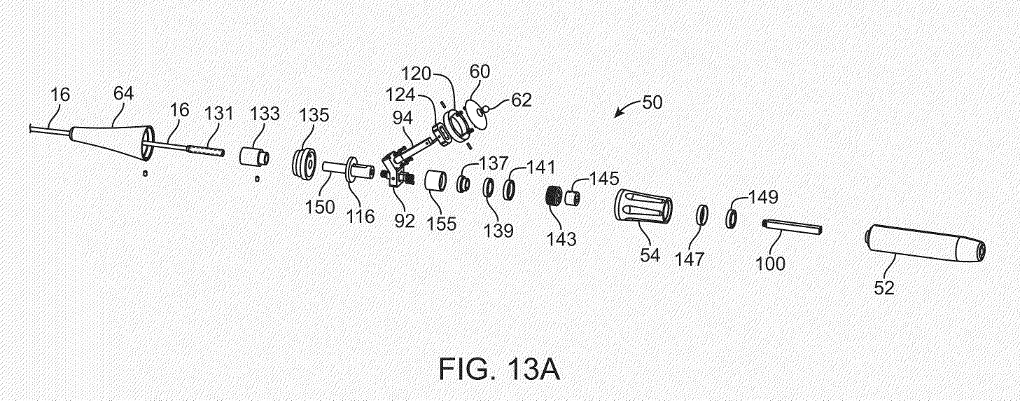

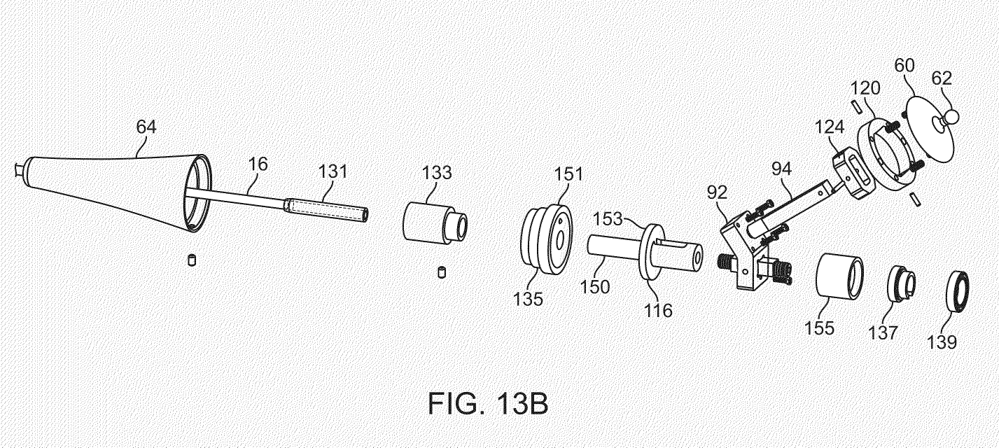

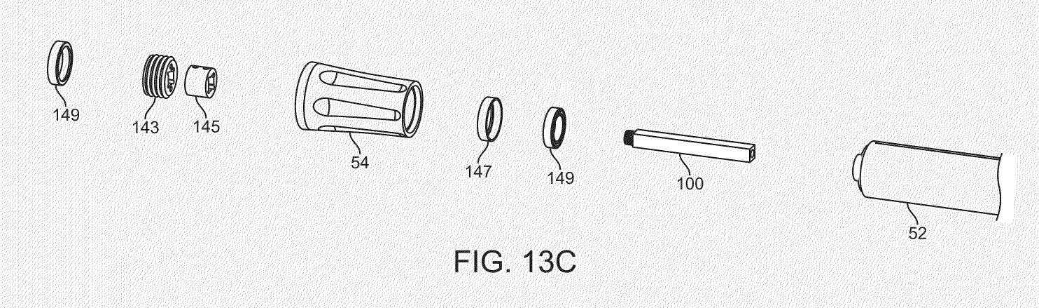

FIG. 13A shows a perspective exploded assembly view of the handle assembly 50 while FIGS. 13B and 13C show detail perspective exploded views of the same handle assembly. As shown, distal steering control 60 having a support member 94 may be supported via platform member 92. As above, a pullwire transition manifold 116 may be positioned proximal to the catheter 16 entrance. Proximal steering control 54 may also be seen rotatably positioned between handle portion 52 and the distal steering control assembly. Because of the design of the handle assembly 50 and the accessibility of the distal steering control 60 to the user, the user may utilize a single hand to operate the handle assembly 50 to control and manipulate the catheter 16 and hood 12 configuration and position within the patient's body. Moreover, the operator may utilize either their right hand or their left hand.

In coupling catheter 16 to the handle assembly 50, a strain relief shaft 131 may be attached to the proximal end of catheter 16 to provide structural support to the catheter and to prevent its kinking relative to the assembly 50. This strain relief shaft 131 may extend at least partially from and within distal handle portion 64. A rotatable plunger housing 135 may fixedly connect to distal handle portion 64 while rotatably positioned via an opening defined through plunger housing 135 over torquing section 150 of transition manifold 116. Plunger housing 135 may further define a surface which interfaces against a portion of transition manifold 116, as described in further detail below. A stop member 133 also defining an opening therethrough for passage of the pullwires may be positioned partially over torquing section 150 distal to plunger housing 135 and affixed to the transition manifold 116 to maintain the plunger housing 135 rotatable positioned over torquing section 150 as well as to maintain the interface between plunger housing 135 and transition manifold 116.

Proximal to platform member 92, a spacer 155 and one or more bearings 137, 139, 141 may be positioned to facilitate the rotation of control 54 with each defining an opening therethrough for accommodating the pullwire from proximal steering control 54. A carriage 143 may define a threaded outer surface and may also define an opening therethrough such that carriage 143 may ride along longitudinal support member 100. Proximal steering control 54 may also define a threaded inner surface which engages the threaded outer surface of carriage 143 in a complementary manner such that rotation of control 54 in a first rotational direction urges carriage 143 to slide in a first direction and rotation of control 54 in an opposite second rotational direction urges carriage 143 to slide in an opposite second direction. The longitudinal support member 100 may have a squared (or otherwise keyed) cross-section which corresponds to a squared (or otherwise keyed) opening defined through carriage 143 to prevent rotation of the carriage 143 when control 54 is rotated. This ensures carriage 143 is translated linearly the along support member 100 to push or pull the appropriate pullwire for controlling the orientation of the catheter distal end.

A stop member 145 may be positioned along or over support member 100 proximally of carriage 143 to limit the travel of carriage 143 along support member 100. Additionally, one or more bearings 147, 149 may also be included to facilitate the rotation of control 54 relative to the handle portion 52.

Turning to the detail exploded assembly view of FIG. 13B, the interface between the surface of plunger housing 135 and transition manifold 116 may be seen. In this particular example, the proximally facing surface of plunger housing 151 may comprise a stop housing 151 which may contain, e.g., a ball-spring element which is urged to project from the housing 151 such as by a spring member, may be positioned along housing 151. The plunger interface surface 153 positioned along the distally facing surface of manifold 116 may define one or more grooves or detents therealong which periodically engage the proximally-urged ball-spring element from the housing 151. In this manner, as distal handle portion 64 is rotated about its longitudinal axis, plunger housing 135 may rotate accordingly to periodically engage the one or more grooves or detents circumferentially along the plunger interface surface 153 to rotate handle portion 64 in a stepped manner which also provides tactile feedback to the operator.

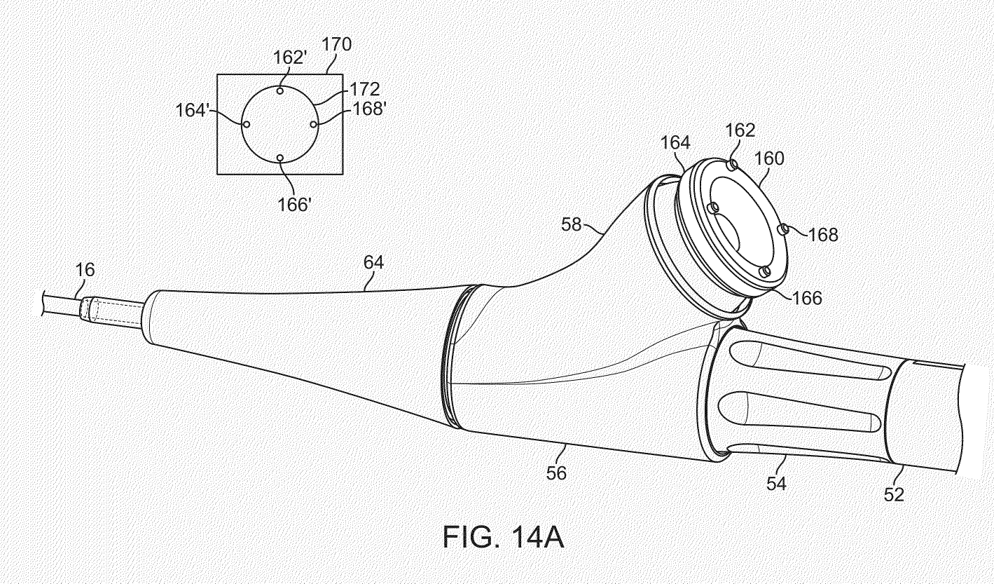

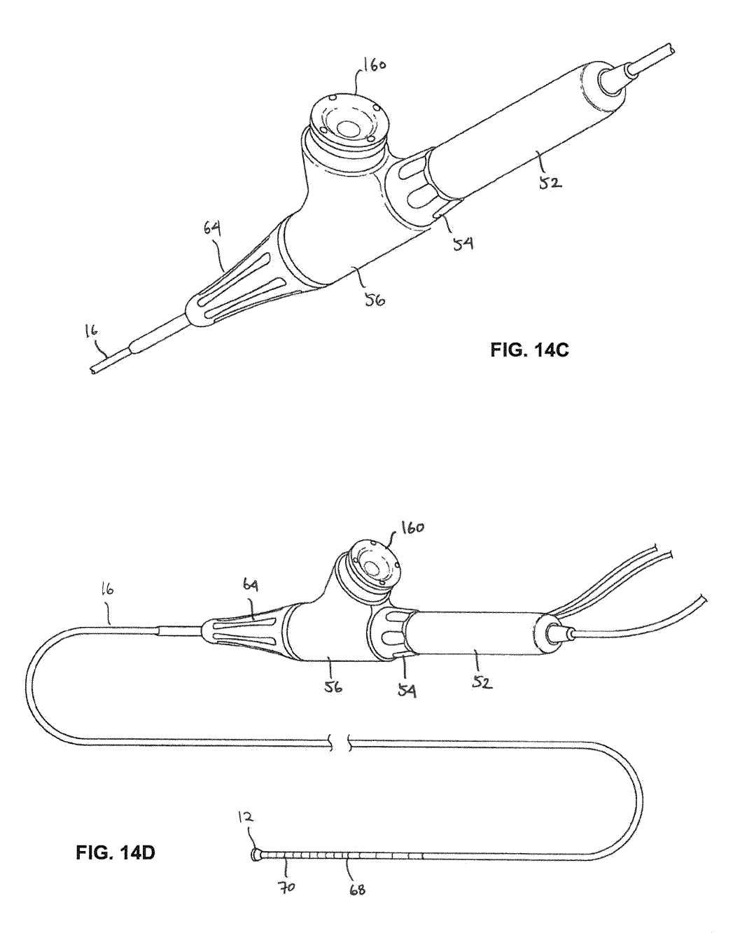

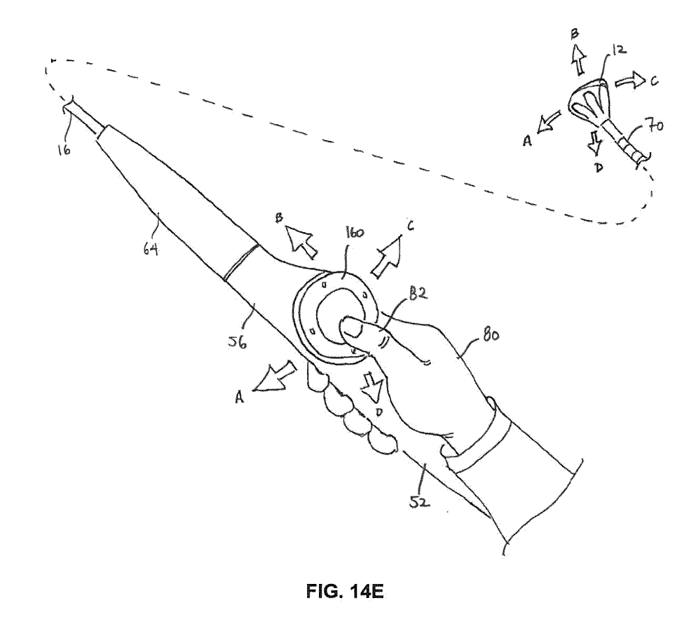

Additionally and/or alternatively, visual indicators positioned directly upon the hood 12 may also be utilized in coordination with corresponding visual indicators positioned upon the distal steering control 160. The hood 12 may have one or more visual indicators marked upon the distal portion of the hood such that the visual image 172 on monitor 170 as captured through the hood 12 may show at least a first directional indicator 162′ along a first portion of the hood, as shown in FIGS. 14A and 14B. In this example, a second directional indicator 164′ and yet a third corresponding third indicator 166′ and fourth directional indicator 168′ may be positioned about a circumference of the hood or hood membrane to represent any number of directions. Handle assembly 50 may thus have one or more directional indicators located directly upon, e.g., distal steering control 160, which is shown in this variation as a circular configuration and which corresponds spatially with the indicators positioned upon the hood or hood membrane. For instance, first directional indicator 162′ on the hood may correspond spatially with first directional indicator 162 on distal steering control 160, second directional indicator 164′ on the hood may correspond spatially with second directional indicator 164 on distal steering control 160, third directional indicator 166′ on the hood may correspond with third directional indicator 166 on distal steering control 160, and fourth directional indicator 168′ on the hood may correspond with fourth directional indicator 168 on distal steering control 160, and so on. Although four directional indicators are shown in this example, fewer than four or more than four may be utilized. Moreover, the location and positioning of the indicators may also be varied, as desired. Additionally, each of the directional indicators may be color-coded by different colors and/or shapes or symbols to distinguish between the different directions.

FIGS. 14C and 14D illustrate additional perspective views of the handle assembly having steering control 160 and also attached to deployment catheter 16 with hood 12 coupled thereon. Also shown are the proximal steering portion 68 (which is controllable by control 54) and distal steering portion 70 (which is controllable by steering control 160) for articulating an orientation of hood 12, as previously described.

In use, the directional indicators as viewed through the hood correspond to the direction the hood may move when the distal steering control 160 is deflected along the position where the corresponding indicator is located. Thus, deflecting distal steering control 160 in a direction of actuation, e.g., along any one or combination of the directional indicators, may articulate the distal steerable section 70 and hood 12 in a corresponding direction of articulation along the directional indicator shown on the hood or hood membrane. For instance, as shown in the perspective illustration of FIG. 14E, an operator may use at least a single finger, such as thumb 82, of a single hand 80 to manipulate the orientation of hood 12 along any one of the directions A, B, C, D (or combination of directions) to articulate hood 12 in a corresponding direction A, B, C, D relative to hood 12. This removes complexity in steering the hood 12, e.g., when the hood 12 is in a retroflexed position, where directions are reversed with respect to the operator. Further details for utilizing directional indicators are described in U.S. patent application Ser. No. 12/499,011 filed Jul. 7, 2009, which has been incorporated herein above.

Turning now to the rotatable distal handle portion, once the distal handle portion has been rotated to re-orient a configuration of the hood within the patient's body, various mechanisms may be utilized for locking and maintaining a position of the rotated handle portion relative to the handle housing 56. The ability to lock and unlock a position of the distal handle portion relative to the housing may allow for the operator to ensure that the re-oriented hood 12 and catheter 16 will maintain its configuration within the patient's body without fear of releasing or becoming displaced inadvertently.

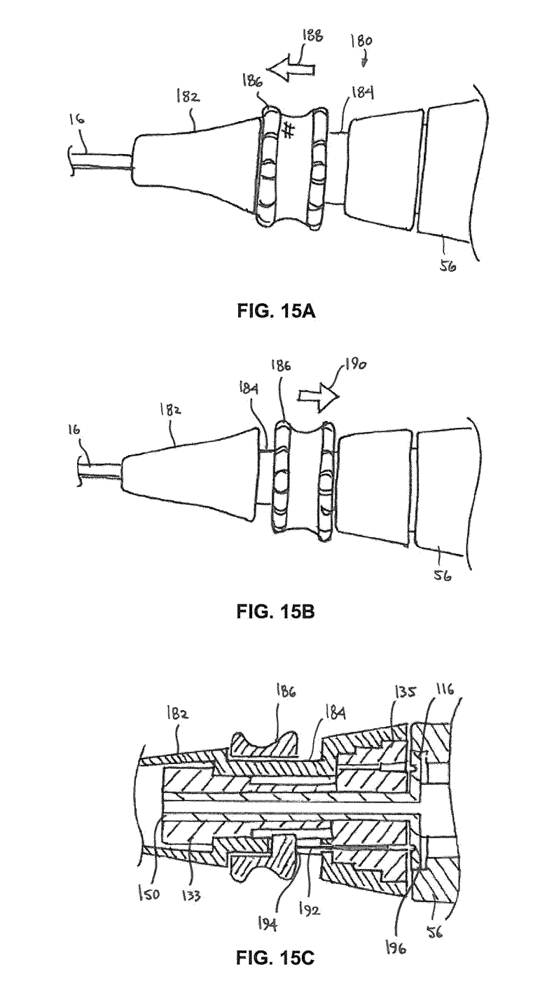

An example of a locking mechanism is illustrated in the side and cross-sectional side views of FIGS. 15A to 15C. The distal housing 182 and distal handle locking assembly 180 may be free to rotate together relative to housing 56 with lock 186 moved along locking guide 184 in a first direction 188 to unlock or enable the rotation of distal housing 182 relative to articulation housing 56, as shown in FIG. 15A. Once the hood 12 and/or catheter 16 has been re-oriented or re-positioned, lock 186 may be moved in a second direction 190 to lock or prohibit the rotation of distal housing 182 relative to articulation housing 56, as shown in FIG. 15B. With lock 186 articulated along the second direction 190, distal housing 182 may be prevented from rotating any further and a position of hood 12 may be maintained. In this example, lock 186 may be urged over locking guide 184 positioned about the torquing section 150 of manifold 116. Lock 186 may comprise a sliding member also coupled at its proximal surface 194 to a first end of an elongate locking member 192 which is also slidably positioned within distal housing 182 and through plunger housing 135, as shown in the cross-sectional side view of FIG. 15C. A second end of the elongate locking member 192 may be partially insertable within one or more corresponding locking grooves or channels 196 defined along a distal surface of manifold 116 such that when lock 186 is in its unlocked position, elongate locking member 192 may be urged distally to de-couple its second end from the groove or channel 196. Likewise, when lock 196 is in its locked position, elongate locking member 192 may be urged proximally to couple its second end into the groove or channel 196 thus preventing any further rotation of distal housing 182 relative to housing 56.

Although specific locking and rotational mechanisms are illustrated and described herein, these are intended to be illustrative and other variations may also be utilized with the devices and methods herein as practicable.

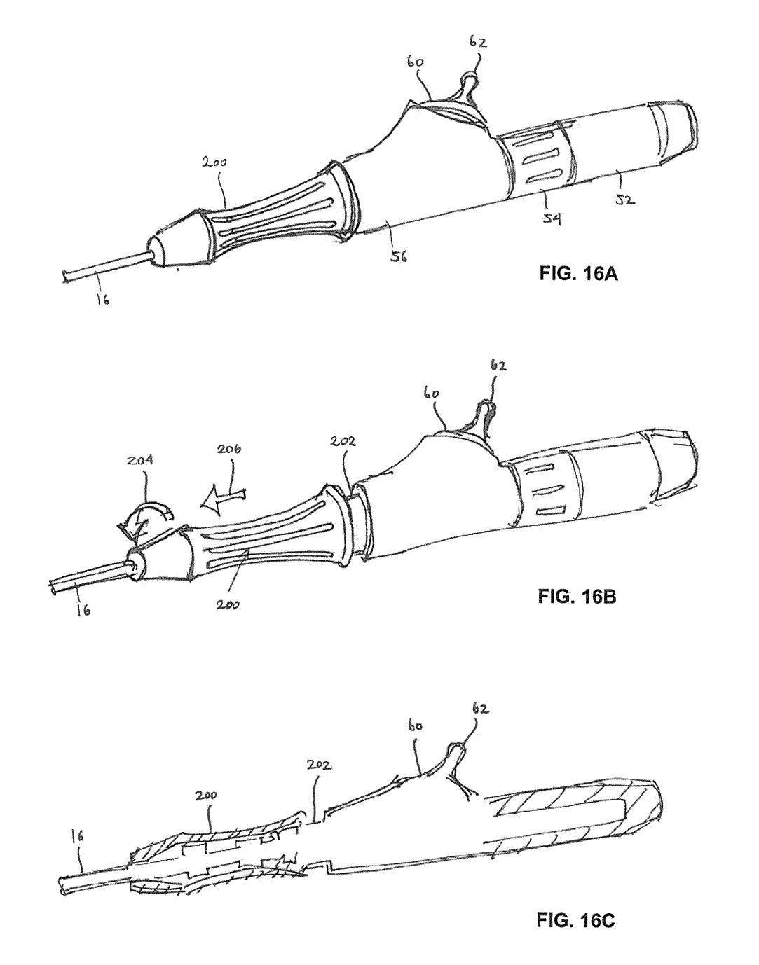

Another example of a locking mechanism for preventing the rotation of the distal handle portion relative to the articulation housing is further shown in the perspective views of FIGS. 16A to 16C. In this example, the catheter handle may comprise a distal handle portion 200 which is slidably detachable from and rotatable relative to the housing 56. Distal handle portion 200 may be slidably coupled along a locking guide portion 202 which extends from housing 56, as shown in the partial cross-sectional view of FIG. 16C, such that handle portion 200 is translatable along guide portion 202 in the first direction 206 to unlock or enable rotation of detachable handle portion 200. Once unlocked, distal handle portion 200 may be rotated, e.g., in a first direction of rotation 204, as shown in FIG. 16B. Once catheter 16 and hood 12 have been re-oriented accordingly, distal handle portion 200 may be retracted to prevent its rotation and to lock and/or maintain an orientation of the catheter 16 and hood 12.

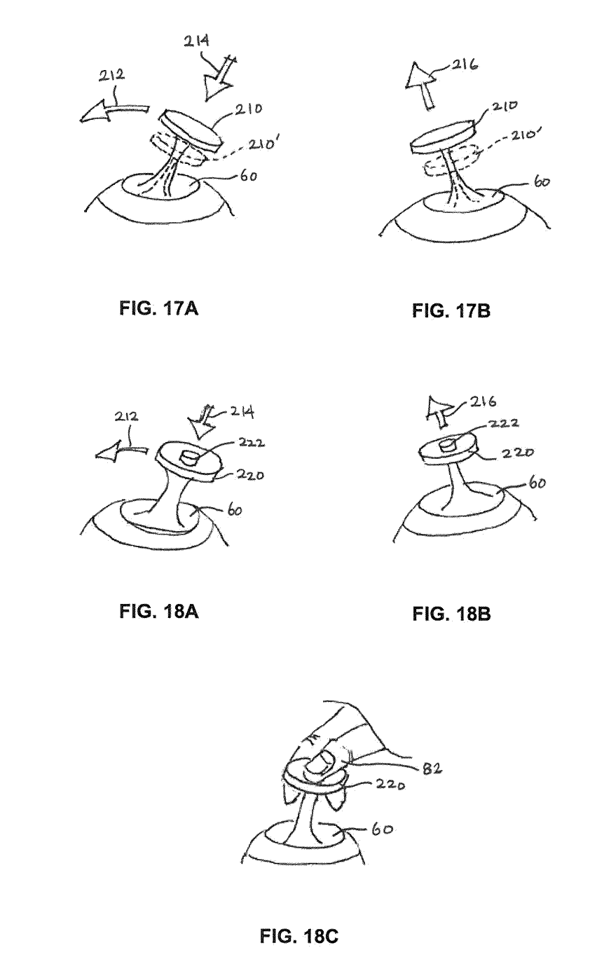

In addition to maintaining a position of the distal handle portion, the articulation control member used to articulate the distal steerable section 70 may also be selectively locked and unlocked to maintain a desired configuration of the distal steerable section 70. An example is illustrated in the perspective views of FIGS. 17A and 17B which show an articulation control 210 extending from control 60 in an unlocked position. When unlocked, articulation control 210 may be moved in any number directions, e.g., in the direction of articulation 212, to correspondingly manipulate the distal steerable section. Once hood 12 has been suitably articulated via distal steerable section 70, the operator may then lock its configuration by depressing articulation control 210, e.g., along its longitudinal axis as indicated by the direction of locking 214, to push articulation control 210 into a locked position 210′, as shown in FIG. 17A. When the operator desires to further articulate distal steerable section 70 to re-orient a position of hood 12, articulation control 210 may be pulled from its locked position 210′ along a direction of unlocking 216 to allow for further manipulation of control 210, as shown in FIG. 17B.

FIGS. 18A to 18C show perspective views of another variation for locking and unlocking articulation control 220 for selectively maintaining a configuration of distal steerable section 70, as described above. In this variation, locking control 222 may be configured as a button positioned upon articulation control 220, as shown. With locking control 222 in an unlocked position, articulation control 220 may be freely manipulated to articulate the distal steerable section. Once articulation control is to be selectively locked, locking control 222 may be depressed in a direction of locking 214 to lock control 220 in position, as shown in FIG. 18A. To allow for the re-orientation of distal steerable section, locking control 222 may be depressed again to disengage it, as shown by the direction of unlocking 216 in FIG. 18B. FIG. 18C illustrates an example of how articulation control 220 may be manipulated and locked or unlocked via a single hand and/or single finger 82.

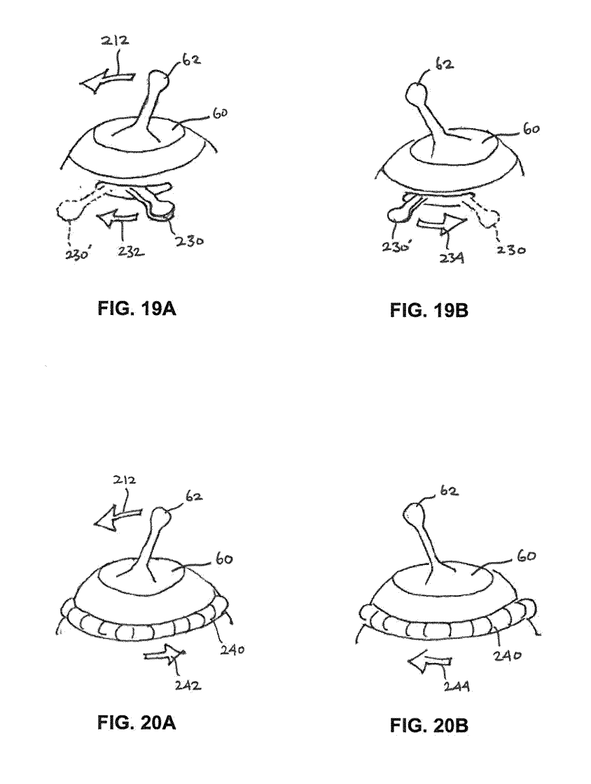

In another variation, locking control 230 may be configured as a projection located adjacent to articulation control 62 and which may be slid into a locked or unlocked position, as shown the perspective views of FIGS. 19A and 19B. With locking control 230 in a first unlocked position, as shown in FIG. 19A, articulation control 62 may be freely manipulated to control the distal steerable section. Locking control 230 may be slid into a locked position 230′ by sliding control 230 in a first direction of locking 232. To unlock a position of articulation control 62, locking control 230 may be slid from its locked position 230′ in a second direction of unlocking 234 to re-position the locking control into its unlocked position 230 where articulation control 62 may be re-manipulated to re-orient the distal steerable section.

In yet another variation, FIGS. 20A and 20B show a variation where locking control 240 may be configured into a rotatable dial positioned circumferentially about the articulation control 62. Circumferential locking control 240 may be rotated in a first direction of locking 242 and/or in a second direction of unlocking 244 to selectively lock and/or unlock articulation control 62 to enable the manipulation and/or maintenance of the distal steerable section.

Although some examples of the locking features may be omitted from particular catheter handles assemblies, it is contemplated that any of the locking features described herein may be utilized with any of the catheter handle assemblies disclosed and as practicable.

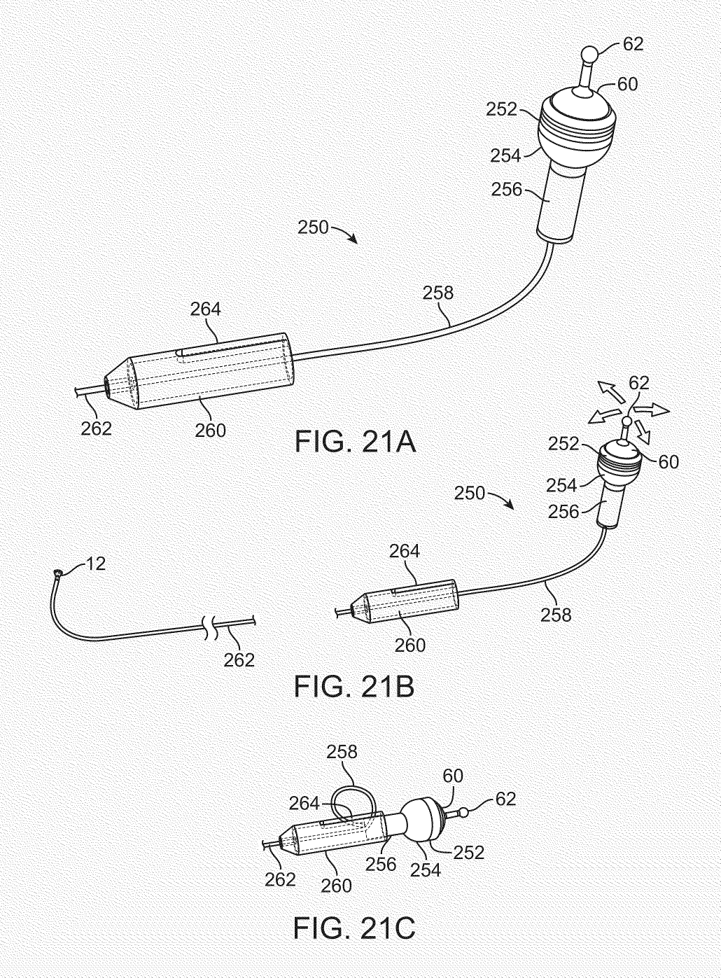

Turning back now to the catheter handle assembly, yet another variation of the catheter handle is shown in the perspective views of FIGS. 21A to 21C, which show a handle assembly 250 comprising a support housing 252 which is removably engagable to a receiving handle 260. Having support housing 252 removable from receiving handle 260 may allow for the operator to initially insert and advance catheter 262 and its distal end effector, such as hood 12, with handle assembly 250 as a complete assembly. If desired, the operator may then disengage support housing 252 from receiving handle 260 to provide unconstrained freedom in articulating the steerable section via distal controller 60, as shown in FIG. 21B, while allowing for catheter 262 to remain stationary relative to the patient's body.

Generally, support housing 252 may comprise an engaging surface 254 and a handle portion 256 which may be tapered and to which a first end of transmission wire bundle 258 (through which one or more pullwires may be passed) may be coupled. The second end of transmission wire bundle 258 may pass into and/or through receiving handle 260 and through catheter 262. To engage support housing 252 to receiving handle 260, handle portion 256 may be inserted into receiving handle 260 while the excess length of transmission wire bundle 258 may be passed into receiving handle 260 or through a slot or channel 264 defined along handle 260, as shown in FIG. 21C. In this manner, support housing 252 may be engaged or disengaged to handle 260 at any time prior to, during, or after a procedure.

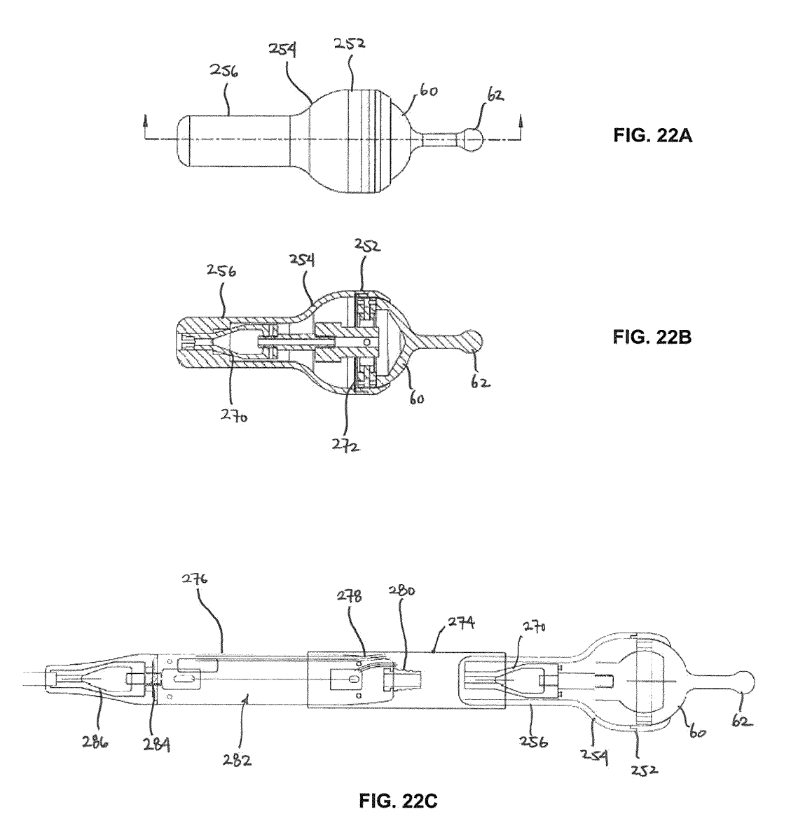

FIGS. 22A to 22C show side and cross-sectional side views of the support housing and receiving handle variations. As shown, support housing 252 may comprise a pullwire transition manifold 270 enclosed within the housing to accommodate one or more pullwires which may be coupled to corresponding pullwire attachments 272 articulatable via articulation control 62. The support housing 252 may be coupled to the receiving handle as previously described or it may be coupled to the variation shown in FIG. 22C. A coupler 274 defining a lumen therethrough may receive the handle portion 256 of support housing 252 and orient the handle relative to a controller assembly 276, which may comprise a fluid lumen 278 for introducing the purging fluid into and/or through the catheter 16 and hood 12 as well as an electrical attachment 280 for coupling to a power supply for providing electrical power to and/or through hood 12. An imaging processor 282 may also be incorporated into controller assembly 276 as well as a one-way steering controller 284 and a pullwire transition manifold 286 for accommodating the one or more pullwires passing into or through the deployment catheter.

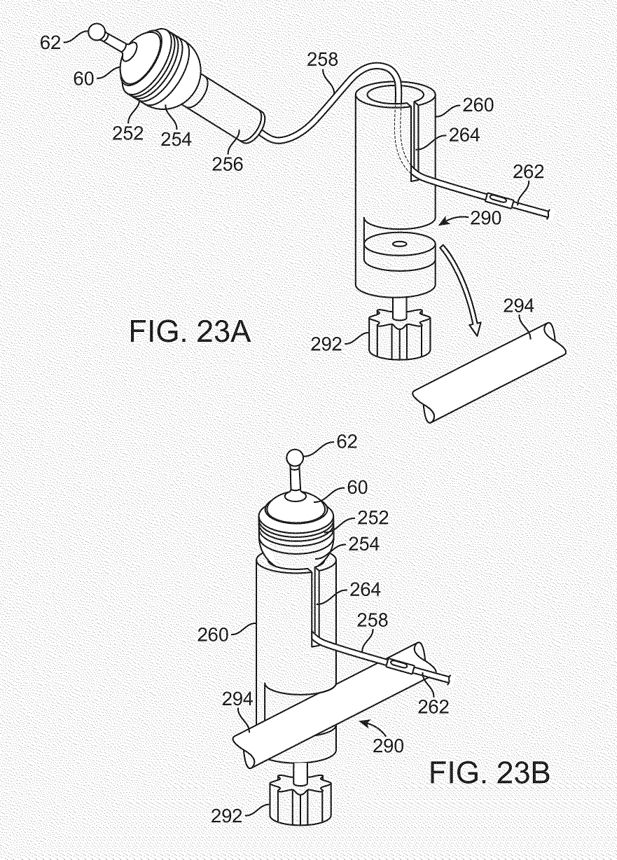

Optionally, receiving handle 260 may also define a locking channel 290 with an actuatable locking mechanism 292 which may be configured to removably lock to a stationary platform such as a bar or rail 294, as shown in FIGS. 23A and 23B. In this configuration, the receiving handle 260 may be locked to, e.g., bar or rail 294, to maintain a stationary relationship between catheter 262 to the patient body while support housing 252 may be removed and freely articulated without transmitting any undesirable movements to the patient.

Although multiple pullwires passing through bundle 258 may be utilized depending upon the number of directions for articulation, four pullwires may be typically utilized. Each of the four pullwires may be terminated to distal steering control 60, as previously described, such that a balanced four-way steering of the distal portion may be accomplished, although manipulating the steering control 60 along various portions of its circumference may yield combinational articulation between the pullwires to result in numerous catheter configurations.

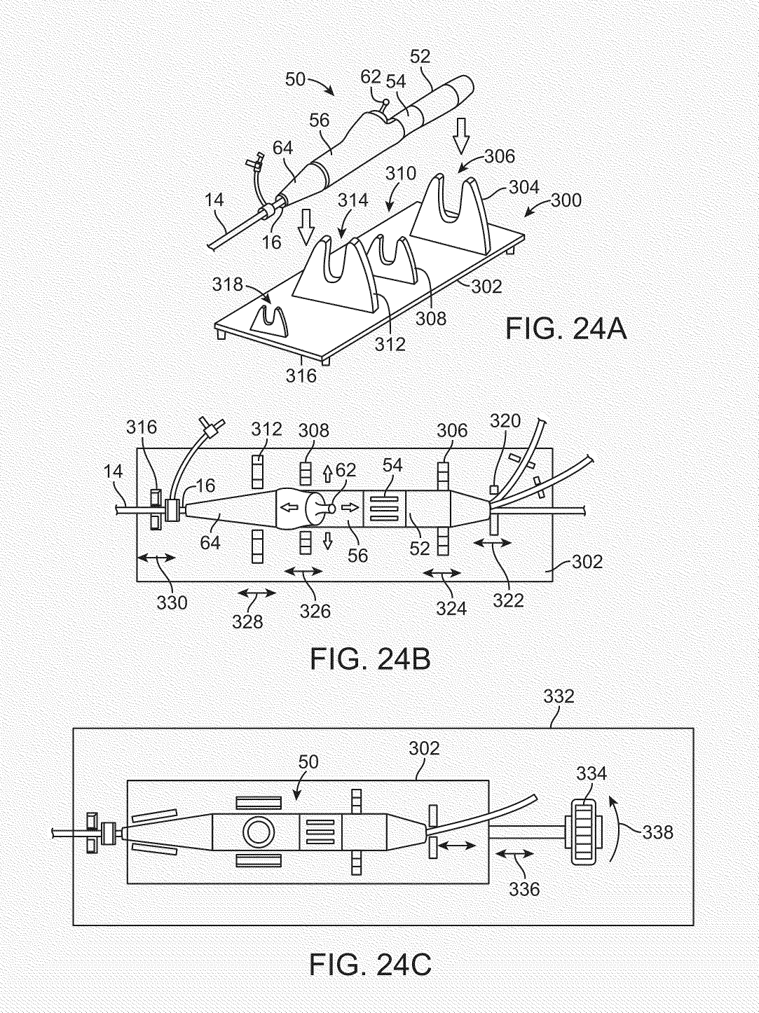

FIGS. 24A to 24C illustrate perspective and top views of another stationary platform which may be used to hold or maintain a position of the catheter handle assembly relative to the patient's body. In this variation, platform 300 may comprise a base 302 from which one or more support members may be movably attached for accommodating the catheter handle and its various mechanisms. The one or more support members may comprise a support element which projects perpendicularly or at an angle from base 302 and which defines an opening appropriately sized to receive a portion of the catheter handle. For example, a proximal handle support 304 may define a receiving opening 306 which is sized to receive a proximal portion of the catheter handle. A mid handle support 308 may be positioned along a mid-portion of base 302 and may define a receiving opening 310 which is sized to receive a mid-portion of the catheter handle while a distal handle support 312 may be positioned at a distal end of the base 302 and may likewise define a receiving opening 314 for receiving the distal handle portion 64. A sheath-catheter support 316 may also be optionally positioned along a distal end of the base 302 as well and define a receiving opening 318 for receiving a proximal end of the outer sheath 14. Sheath-catheter support 316 may also be utilized to lock and/or maintain a longitudinal position of the outer sheath 14 relative to the deployment catheter 16 over which it may be positioned.

As further illustrated in the top view of FIG. 24B, a cable support 320 may also be included along base 302 for optionally separating and/or securing any of the cables which may extend from the handle assembly. Moreover, any one or all of the supports may be adjustable longitudinally (and/or transversely) relative to base 302 to provide for adjustments or to accommodate variations in the handle assembly, as illustrated by each of the arrows 322, 324, 326, 328, 330 indicating the adjustability of the appropriate support member.

Additionally and/or alternatively, the entire base 302 may be adjustably positioned upon another stationary platform 332 to allow for the adjustment of the handle assembly (and base 302) relative to the patient's body. As shown in the top view of FIG. 24C, an actuator 334 may be coupled to the base 302 upon which the handle assembly 50 is attached to enable the longitudinal adjustment of the attached handle, as indicated by the direction of translation 336. Additionally, the entire base 302 and handle assembly 50 may also be rotatably adjustable, as indicated by the direction of rotation 338, to allow for the angular adjustment of the assembly relative to the patient, if so desired.

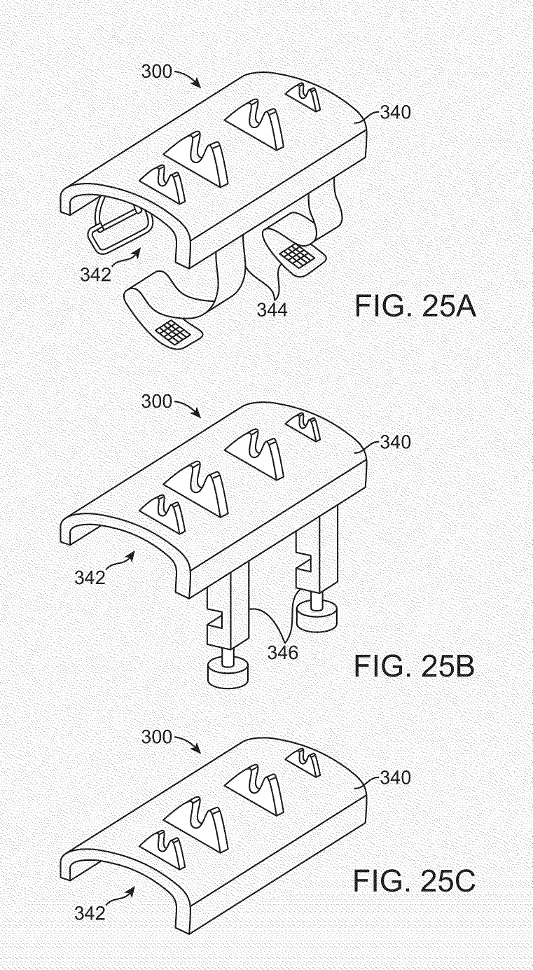

An alternative variation is shown in the perspective view of FIG. 25A, which shows a platform 300 which may be attached directly to the patient's body, such as along the leg inferior to a catheter entry location like the patient's groin. In this variation, platform 300 may comprise a curved platform 340 which defines a receiving channel 342 for accommodating the patient's limb and one or more straps 344 for securing the platform onto the patient's body. Another variation is illustrated in FIG. 25B which shows a perspective view of a curved platform 340 extending from one or more platform locking members 346 which may be attached to the bed or railing in proximity to the patient's body. Yet another variation is shown in the perspective view of FIG. 25C, which illustrates a curved platform 340 which may be simply laid upon the patient's body with the handle assembly secured thereto.

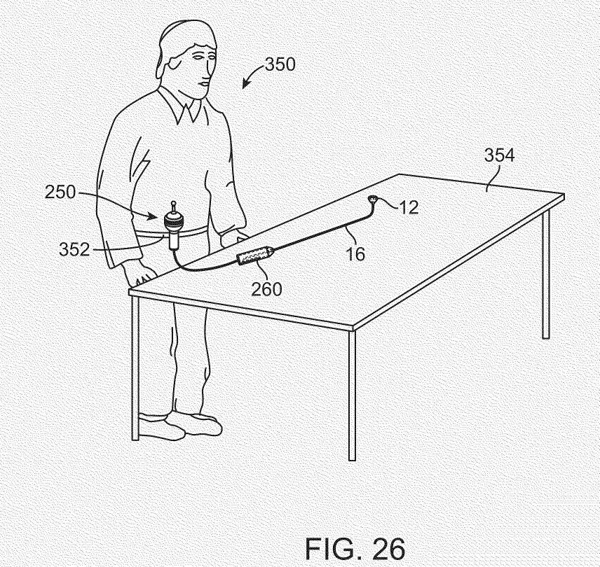

In yet another variation shown in the perspective view of FIG. 26, the catheter handle assembly may be simply attached temporarily to the operator 350 via a belt attachment 352. Accordingly, the receiving handle 260 may positioned along or secured to the table 354 upon which the patient lies thus freeing both of the operator's hands at least temporarily.

The applications of the disclosed invention discussed above are not limited to certain treatments or regions of the body, but may include any number of other applications as well. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A catheter control handle, comprising:
a housing having an elongate catheter extending therefrom;
a proximal steering control positioned along the housing and in communication with a proximal steerable section which is articulatable by the proximal steering control and located near a distal end of the catheter; and,
a distal steering control positioned along the housing such that the distal steering control is operable via a single finger and which is in communication with a distal steerable section which is articulatable by the distal steering control and located distal to the proximal steerable section.

2. The handle of claim 1 wherein the housing comprises a distal handle portion coupling the housing to the catheter which is attached to the distal handle portion.

3. The handle of claim 2 wherein the distal handle portion is rotatable about a longitudinal axis of the housing relative to the housing.

4. The handle of claim 2 wherein the distal handle portion is tapered.

5. The handle of claim 3 wherein the distal handle portion is rotatable up to 720 degrees or more.

6. The handle of claim 3 further comprising a locking mechanism movable between a locked and unlocked position which selectively locks a position of the distal handle portion relative to the housing.

7. The handle of claim 2 further comprising a stop which limits rotation of the distal handle portion.

8. The handle of claim 1 wherein the proximal steering control comprises a rotational control element positioned along the handle.

9. The handle of claim 1 wherein the proximal steerable section is constrained to articulate within a single plane.

10. The handle of claim 1 wherein the distal steering control is angled relative to a longitudinal axis of the housing.

11. The handle of claim 10 wherein the distal steering control is angled such that the distal steering control is off-set relative to the proximal steering control.

12. The handle of claim 1 wherein the distal steerable section is articulatable in at least two or more planes.

13. The handle of claim 1 wherein the distal steering control is coupled to at least one pullwire connected to the distal steerable section.

14. The handle of claim 1 further comprising a locking mechanism positioned along the distal steering control and movable between a locked and unlocked position which selectively locks a position of the distal steerable section.

15. The handle of claim 1 further comprising a hood with a hood membrane extending from a distal end of the catheter.

16. The handle of claim 15 wherein the hood or a hood membrane define one or more positional indicators located around a periphery such that the one or more positional indicators correspond to one or more indicators located around a periphery of the distal steering control.

17. The handle of claim 1 further comprising a platform having one or more support members each defining a receiving portion sized to accommodate the housing.

18. The handle of claim 17 wherein a position of the platform relative to a patient body is adjustable.

19. The handle of claim 1 wherein the distal steering control is operable via multiple additional fingers.

20. A method for controlling a catheter, comprising:
maintaining a handle housing and catheter extending from the handle in a first orientation;
actuating a proximal steering control positioned along the handle via at least one finger such that a proximal steerable section located near a distal end of the catheter articulates within a single plane; and,
actuating a distal steering control positioned along the handle in a first direction via a single finger such that a distal steerable section of the catheter articulates in a corresponding first direction.

21. The method of claim 20 further comprising rotating a distal handle portion of the handle housing to reorient the catheter from a first orientation to a second orientation while maintain a position of a remaining portion of the handle relative to a user.

22. The method of claim 21 further comprising actuating the distal steering control in the first direction via the single finger such that the distal steerable section of the catheter articulates in the corresponding first direction.

23. The method of claim 21 further comprising locking a position of the distal handle portion relative to the remaining portion of the handle.

24. The method of claim 20 further comprising advancing the catheter intravascularly into a patient body prior to actuating the proximal steering control.

25. The method of claim 20 wherein maintaining comprises positioning the distal steerable section within a heart of a patient.

26. The method of claim 20 further comprising locking a position of the distal steering control such that a configuration of the distal steerable section is maintained.

27. The method of claim 20 wherein manipulating comprises further manipulating the distal steering control along at least a second direction such that the distal steerable section articulates in a corresponding second direction.

28. The method of claim 20 further comprising visualizing through a hood projecting from a distal end of the catheter while maintaining the handle housing.

29. The method of claim 20 further comprising:
visually identifying at least a first positional indicator located along the hood corresponding to a desired direction of articulation; and
manipulating the distal steering control toward a first positional indicator located on the distal steering control which corresponds to the first positional indicator located along the hood.

30. The method of claim 20 further comprising adjusting a position of the handle housing relative to a patient body via an adjustable platform.

31. The method of claim 20 wherein actuating a distal steering control comprises actuating via two or more fingers.

32. A catheter control handle, comprising:
a housing having an elongate catheter extending therefrom;
a distal handle portion coupling the housing to the catheter which is attached to the distal handle portion and which is rotatable about a longitudinal axis of the housing relative to the housing;
a proximal steering control positioned along the housing and in communication with a proximal steerable section which is articulatable by the proximal steering control and located near a distal end of the catheter; and,
a distal steering control positioned along the housing such that the distal steering control is operable via a single finger and which is in communication with a distal steerable section which is articulatable by the distal steering control and located distal to the proximal steerable section.

33. The handle of claim 32 wherein the distal handle portion is tapered.

34. The handle of claim 32 wherein the distal handle portion is rotatable up to 720 degrees or more.

35. The handle of claim 32 further comprising a stop which limits rotation of the distal handle portion.

36. The handle of claim 32 wherein the proximal steering control comprises a rotational control element positioned along the handle.

37. The handle of claim 32 wherein the proximal steerable section is constrained to articulate within a single plane.

38. The handle of claim 32 wherein the distal steering control is angled relative to a longitudinal axis of the housing.

39. The handle of claim 38 wherein the distal steering control is angled such that the distal steering control is off-set relative to the proximal steering control.

40. The handle of claim 32 wherein the distal steerable section is articulatable in at least two or more planes.

41. The handle of claim 32 wherein the distal steering control is coupled to one or more pullwires connected to the distal steerable section.

42. The handle of claim 41 further comprising an isolation coil positioned about each one of the one or more pullwires.

43. The handle of claim 42 wherein the housing or distal handle defines a space within which the one or more pullwires are rotatable upon one another when the distal handle portion is rotated relative to the housing.

44. The handle of claim 32 further comprising a hood with a hood membrane extending from a distal end of the catheter.

45. The handle of claim 44 wherein the hood or a hood membrane define one or more positional indicators located around a periphery such that the one or more positional indicators correspond to one or more indicators located around a periphery of the distal steering control.

46. The handle of claim 32 further comprising a locking mechanism movable between a locked and unlocked position which selectively locks a position of the distal handle portion relative to the housing.

47. The handle of claim 32 further comprising a locking mechanism positioned along the distal steering control and movable between a locked and unlocked position which selectively locks a position of the distal steerable section.

48. The handle of claim 32 further comprising a platform having one or more support members each defining a receiving portion sized to accommodate the housing.

49. The handle of claim 48 wherein a position of the platform relative to a patient body is adjustable.

50. The handle of claim 32 wherein the distal steering control is operable via multiple additional fingers.

51. A catheter control handle, comprising:
a housing having an elongate catheter extending therefrom; and
a steering control positioned along the housing whereby the steering control is operable via a single finger to articulate a steerable section positioned near or at a distal end of the catheter.

52. The handle of claim 51 wherein the housing comprises a distal handle portion coupling the housing to the catheter which is attached to the distal handle portion.

53. The handle of claim 52 wherein the distal handle portion is rotatable about a longitudinal axis of the housing relative to the housing.

54. The handle of claim 52 wherein the distal handle portion is tapered.

55. The handle of claim 53 wherein the distal handle portion is rotatable up to 720 degrees or more.

56. The handle of claim 52 further comprising a stop which limits rotation of the distal handle portion.

57. The handle of claim 51 wherein the steering control is angled relative to a longitudinal axis of the housing.

58. The handle of claim 57 wherein the steering control is angled to be off-set relative to a proximal steering control positioned along the handle.

59. The handle of claim 51 wherein the steerable section is articulatable in at least two or more planes by the steering control.

60. The handle of claim 51 wherein the steering control is coupled to at least one pullwire connected to the steerable section.

61. The handle of claim 51 further comprising a hood with a hood membrane extending from a distal end of the catheter.

62. The handle of claim 61 wherein the hood or a hood membrane define one or more positional indicators located around a periphery such that the one or more positional indicators correspond to one or more indicators located around a periphery of the distal steering control.

63. The handle of claim 51 further comprising a locking mechanism movable between a locked and unlocked position which selectively locks a position of the distal handle portion relative to the housing.

64. The handle of claim 51 further comprising a locking mechanism positioned along the distal steering control and movable between a locked and unlocked position which selectively locks a position of the distal steerable section.

65. The handle of claim 51 further comprising a platform having one or more support members each defining a receiving portion sized to accommodate the housing.

66. The handle of claim 65 wherein a position of the platform relative to a patient body is adjustable.
